SEQUENCE LISTING (69 sequences)

SEQ ID NO: 1 - DNA, 1779 bp, Arabidopsis thaliana
  CDS (1)..(1779)
atgaattccgttcagcttccgacggcgcaatcctctctccgtagccacattcaccgtccatcaaaaccagtggtcggattcactcacttctcctcccgttctcggatcgcagtggcggttctgtcccgagatgaaacatctatgactccaccgcctccaaagcttcctttaccacgtcttaaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgcgttacttgatgggaggaagatctactgttggagacgaggttctatgccgattcacctttcccgagagacctggtgctctaatgaacttcttggactctttcagtccacggtggaacatcacccttttccattaccgtggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactga

SEQ ID NO: 2 - protein, 592 aa, Arabidopsis thaliana
MNSVQLPTAQSSLRSHIHRPSKPVVGFTHFSSRSRIAVAVLSRDETSMTPPPPKLPLPRLKVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLRYLMGGRSTVGDEVLCRFTFPERPGALMNFLDSFSPRWNITLFHYRGQGETGANVLVGIQVPEQEMEEFKNRAKALGYDYFLVSDDDYFKLLMH

SEQ ID NO: 3 - DNA, 1779 bp, Arabidopsis thaliana
  CDS (1)..(1779)
atgaattccgttcagcttccgacggcgcaatcctctctccgtagccacattcaccgtccatcaaaaccagtggtcggattcactcacttctcctcccgttctcggatcgcagtggcggttctgtcccgagatgaaacatctatgactccaccgcctccaaagcttcctttaccacgtcttaaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactga

SEQ ID NO: 4 - protein, 592 aa, Arabidopsis thaliana
MNSVQLPTAQSSLRSHIHRPSKPVVGFTHFSSRSRIAVAVLSRDETSMTPPPPKLPLPRLKVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMH

SEQ ID NO: 5 - DNA, 1830 bp, Arabidopsis thaliana
  CDS (1)..(1830)
atgggcgagctcggtacccggggatcctctagaactagtggatcccccgggctgcaggaattcggcacgaggacggcgcaatcctctctccgtagccacattcaccgtccatcaaaaccagtggtcggattcactcacttctcctcccgttctcggatcgcagtggcggttctgtcccgagatgaaacatctatgactccaccgcctccaaagcttcctttaccacgtcttaaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactga

SEQ ID NO: 6 - protein, 609 aa, Arabidopsis thaliana
MGELGTRGSSRTSGSPGLQEFGTRTAQSSLRSHIHRPSKPVVGFTHFSSRSRIAVAVLSRDETSMTPPPPKLPLPRLKVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMH

SEQ ID NO: 7 - DNA, 1509 bp, Arabidopsis thaliana
  CDS (1)..(1509)
gaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactga

SEQ ID NO: 8 - protein, 502 aa, Arabidopsis thaliana
EAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMH

SEQ ID NO: 9 - DNA, 1620 bp, Arabidopsis thaliana
  CDS (1)..(1620)
aagcttcctttaccacgtcttaaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactga

SEQ ID NO: 10 - protein, 539 aa, Arabidopsis thaliana
KLPLPRLKVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMH

SEQ ID NO: 11 - DNA, 1599 bp, Arabidopsis thaliana
  CDS (1)..(1599)
aaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactga

SEQ ID NO: 12 - protein, 532 aa, Arabidopsis thaliana
KVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMH

SEQ ID NO: 13 - DNA, 720 bp, Arabidopsis thaliana
  CDS (1)..(720)
tcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcac

SEQ ID NO: 14 - protein, 240 aa, Arabidopsis thaliana
SIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMH

SEQ ID NO: 15 - DNA, 81 bp, Arabidopsis thaliana
  CDS (1)..(81)
gtcaatctcactaccagtgacttagtgaaagatcacctgtgttacttgatgggaggaagatctactgttggagacgaggtt

SEQ ID NO: 16 - protein, 27 aa, Arabidopsis thaliana
VNLTTSDLVKDHLCYLMGGRSTVGDEV

SEQ ID NO: 17 - DNA, 75 bp, Arabidopsis thaliana
  CDS (1)..(75)
tggaacatcacccttttccattaccatggacagggtgagacgggcgcgaatgtgctggtcgggatccaagtcccc

SEQ ID NO: 18 - protein, 25 aa, Arabidopsis thaliana
WNITLFHYHGQGETGANVLVGIQVP

SEQ ID NO: 19 - DNA, 1638 bp, Arabidopsis thaliana
  CDS (1)..(1638)
atgactccaccgcctccaaagcttcctttaccacgtcttaaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactga

SEQ ID NO: 20 - protein, 545 aa, Arabidopsis thaliana
MTPPPPKLPLPRLKVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMH

SEQ ID NO: 21 - protein, 19 aa, Arabidopsis thaliana
VNLTTSDLVKDHLRYLMGG

SEQ ID NO: 22 - protein, 19 aa, Artificial Sequence
  Synthetic
XXXXXXXXXXXHXXXXXGG

SEQ ID NO: 23 - protein, 19 aa, Artificial Sequence
  Synthetic
XXXXXXXXXXXHXCXXXGG

SEQ ID NO: 24 - protein, 19 aa, Arabidopsis thaliana
VNLTTSDLVKDHLCYLMGG

SEQ ID NO: 25 - protein, 19 aa, Arabidopsis thaliana
WNITLFHYHGQGETGANVL

SEQ ID NO: 26 - protein, 590 aa, Cicer arietinum
MLSTSTTNSSILPFRSRASSSTFIARPPANFNSIFTTSVRVFPISMSRYCVFPHTWERDHNVPGVPGVLRKVVPAAPIKNKPTCADSDELPEYLRDVLRSPVYDVVVESPVELTERLSDRLGVNFYVKREDRQRVFSFKLRGPYNMMSSLSHEEIDKGVITASAGNHAQGVPFPFPGRRLKCVAKIVMPTTTPNIKLDGVRALGADVVLWGHTFDEAKTHAVELCEKDGLRTIPPFEDPAVIKGQGTIGSEINRQIKRIDAVFVPVGGGGLIAGVAAFFKQIAPQTKIIVVEPYDAASMALSVHAEHRAKLSNVDTFADGATVAVIGEYTFARCQDVVDAMVLVANDGIGAAIKDVFDEGRNIVETSGAAGIAGMYCEMYRIKNDNMVGIVSGANMNFRKLHKVSELAVLGSGHEALLGTYMPGQKGCFKTMAGLVHGSLSFTEITYRFTSHRRSILVLMLKLEPWRYIEKMIEMMKYSGVTVLNISHNELAVIHGKHLVGGSAKVSDEVFVEFIIPEKADLKKFLEVLSPHWNLTLYRYRNQGDLKATILMVIASFLCEIVIRKNQIDDLGYPYEIDQYNDAFNLAVTE

SEQ ID NO: 27 - protein, 595 aa, Lycopersicon esculentum
MEFLCLAPTRSFSTNPKLTKSIPSDHTSTTSRIFTYQNMRGSTMRPLALPLKMSPIVSVPDITAPVENVPAILPKVVPGELIVNKPTGGDSDELFQYLVDILASPVYDVAIESPLELAEKLSDRLGVNFYIKREDKQRVFSFKLRGAYNMMSNLSREELDKGVITASAGNHAQGVALAGQRLNCVAKIVMPTTTPQIKIDAVRALGGDVVLYGKTFDEAQTHALELSEKDGLKYIPPFDDPGVIKGQGTIGTEINRQLKDIHAVFIPVGGGGLIAGVATFFKQIAPNTKIIGVEPYGAASMTLSLHEGHRVKLSNVDTFADGVAVALVGEYTFAKCQELIDGMVLVANDGISAAIKDVYDEGRNILETSGAVAIAGAAAYCEFYKIKNENIVAIASGANMDFSKLHKVTELAGLGSGKEALLATFMVEQQGSFKTFVGLVGSLNFTELTYRFTSERKNALILYRVNVDKESDLEKMIEDMKSSNMTTLNLSHNELVVDHLKHLVGGSANISDEIFGEFIVPEKAETLKTFLDAFSPRWNITLCRYRNQGDINASLLMGFQVPQAEMDEFKNQADKLGYPYELDNYNEAFNLVVSE

SEQ ID NO: 28 - protein, 359 aa, Solanum tuberosum
PFDAPGVIKGQGTIGTEINRQLKDIHAVFVPVGGGGLISGVAAYFTQVAPHTKIIGVEPYGAASMTLSLYEGHRVKLENVDTFADGVAVALVGEYTFAKCQELIDGMVLVRNDGISAAIKDVYDEGRNILETSGAVAIAGAAAYCEFYNIKNENIVAIASGANMDFSKLHKVTELAELGSDNEALLATFMIEQPGSFKTFAKLVGSMNITEVTYRFTSERKEALVLYRVDVDEKSDLEEMIKKLNSSNMKTFNFSHNELVAEHIKHLVGGSASISDEIFGEFIFPEKAGTLSTFLEAFSPRWNITLCRYRDQGDINGNVLVGFQVPQSEMDEFKSQADGLGYPYELDNSNEAFNIVVAE

SEQ ID NO: 29 - protein, 576 aa, Saccharomyces cerevisiae
MSATLLKQPLCTVVRQGKQSKVSGLNLLRLKAHLHRQHLSPSLIKLHSELKLDELQTDNTPDYVRLVLRSSVYDVINESPISQGVGLSSRLNTNVILKREDLLPVFSFKLRGAYNMIAKLDDSQRNQGVIACSAGNHAQGVAFAAKHLKIPATIVMPVCTPSIKYQNVSRLGSQVVLYGNDFDEAKAECAKLAEERGLTNIPPFDHPYVIAGQGTVAMEILRQVRTANKIGAVFVPVGGGGLIAGIGAYLKRVAPHIKTIGVETYDAATLHNSLQRNQRTPLPVVGTFADGTSVRMIGEETFRVAQQVVDEVVLVNTDEICAAVKDIFEDTRSIVEPSGALSVAGMKKYISTVHPEIDHTKNTYVPILSGANMNFDRLRFVSERAVLGEGKEVFMLVTLPDVPGAFKKMQKIIHPRSVTEFSYRYNEHRHESSSEVPKAYIYTSFSVVDREKEIKQVMQQLNALGFEAVDISDNELAKSHGRYLVGGASKVPNERIISFEFPERPGALTRFLGGLSDSWNLTLFHYRNHGADIGKVLAGISVPPRENLTFQKFLEDLGYTYHDETDNTVYQKFLKY

SEQ ID NO: 30 - protein, 360 aa, Saccharomyces cerevisiae
MSIVYNKTPLLRQFFPGKASAQFFLKYECLQPSGSFKSRGIGNLIMKSAIRIQKDGKRSPQVFASSGGNAGFAAATACQRLSLPCTVVVPTATKKRMVDKIRNTGAQVIVSGAYWKEADTFLKTNVMNKIDSQVIEPIYVHPFDNPDIWEGHSSMIDEIVQDLKSQHISVNKVKGIVCSVGGGGLYNGIIQGLERYGLADRIPIVGVETNGCHVFNTSLKIGQPVQFKKITSIATSLGTAVISNQTFEYARKYNTRSVVIEDKDVIEPCLKYTHQFNMVIEPACGAALHLGYNTKILENALGSKLAADDIVIIIACASSSNTIKDLEEALDSMRKKDTPVIEVADNFIFPEKNIVNLKSA

SEQ ID NO: 31 - protein, 514 aa, Salmonella typhimurium
MAESQPLSVAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKSLIVMPKATADIKVDAVRGFGGEVLLHGANFDEAKAKAIELAQQQGFTWVPPFDHPMVIAGQGTLALELLQQDSHLDRVFVPVGGGGLAAGVAVLIKQLMPQIKVIAVEAEDSACLKAALEAGHPVDLPRVGLFAEGVAVKRIGDETFRLCQEYLDDIITVDSDAICAAMKDLFEDVRAVAEPSGALALAGMKKYIAQHNIRGERLAHVLSGANVNFHGLRYVSERCELGEQREALLAVTIPEEKGSFLKFCQLLGGRMVTEFNYRFADAKNACIFVGVRVSQGLEERKEIITQLCDGGYSVVDLSDDEMAKLHVRYMVGGRPSKPLQERLYSFEFPESPGALLKFLHTLGTHWNISLFHYRSHGTDYGRVLAAFELGDHEPDFETRLHELGYECHDESNNPAFRFFLAG

SEQ ID NO: 32 - protein, 514 aa, Escherichia coli [sequence omitted]

SEQ ID NO: 33 - protein, 329 aa, Escherichia coli
MHITYDLPVAIDDIIEAKQRLAGRIYKTGMPRSNYFSERCKGEIFLKFENMQRTGSFKIRGAFNKLSSLTDAEKRKGVVACSAGNHAQGVSLSCAMLGIDGKVVMPKGAPKSKVAATCDYSAEVVLHGDNFNDTIAKVSEIVEMEGRIFIPPYDDPKVIAGQGTIGLEIMEDLYDVDNVIVPIGGGGLIAGIAVAIKSINPTIRVIGVQSENVHGMAASFHSGEITTHRTTGTLADGCDVSRPGNLTYEIVRELVDDIVLVSEDEIRNSMIALIQRNKVVTEGAGALACAALLSGKLDQYIQNRKTVSIISGGNIDLSRVSQITGFVDA

SEQ ID NO: 34 - protein, 12 aa, Artificial Sequence
  Synthetic
FSFKLRGAYNMM

SEQ ID NO: 35 - protein, 12 aa, Artificial Sequence
  Synthetic
FSFKLRGAYNMM

SEQ ID NO: 36 - protein, 8 aa, Artificial Sequence
  Synthetic
LRGAYNMM

SEQ ID NO: 37 - DNA, 28 bp, Artificial Sequence
  Synthetic
gggaattcnngnggngcntanaanatga

SEQ ID NO: 38 - protein, 12 aa, Artificial Sequence
  Synthetic
AVGAILGGGGVP

SEQ ID NO: 39 - protein, 12 aa, Artificial Sequence
  Synthetic
AVAGILGGGGVP

SEQ ID NO: 40 - protein, 7 aa, Artificial Sequence
  Synthetic
ILGGGGV

SEQ ID NO: 41 - DNA, 28 bp, Artificial Sequence
  Synthetic
ataagcttatnanaccnccnccnccnac

SEQ ID NO: 42 - protein, 19 aa, Arabidopsis thaliana
VNLTTSDLVKDHLRYLMGG

SEQ ID NO: 43 - protein, 19 aa, Escherichia coli
VNLSDDEMAKLHVRYMVGG

SEQ ID NO: 44 - protein, 19 aa, Salmonella typhimurium
VNLSDDEMAKLHVRYMVGG

SEQ ID NO: 45 - protein, 19 aa, Saccharomyces cerevisiae
VDISDNELAKSHGRYLVGG

SEQ ID NO: 46 - protein, 19 aa, Lycopersicon esculentum
LNLSHNELVVDHLKHLVGG

SEQ ID NO: 47 - protein, 19 aa, Cicer arietinum
LNISHNELAVIHGKHLVGG

SEQ ID NO: 48 - DNA, 15 bp, Arabidopsis thaliana
cacctgcgttacttg

SEQ ID NO: 49 - DNA, 15 bp, Arabidopsis thaliana
cacctgtgttacttg

SEQ ID NO: 50 - protein, 19 aa, Arabidopsis thaliana
WNITLFHYRGQGETGANVL

SEQ ID NO: 51 - protein, 19 aa, Escherichia coli
WNISLFHYRSHGTDYGRVL

SEQ ID NO: 52 - protein, 19 aa, Salmonella typhimurium
WNISLFHYRSHGTDYGRVL

SEQ ID NO: 53 - protein, 19 aa, Saccharomyces cerevisiae
WNLTLFHYRNHGADIGKVL

SEQ ID NO: 54 - protein, 19 aa, Lycopersicon esculentum
WNITLCRYRNQGDINASLL

SEQ ID NO: 55 - protein, 19 aa, Cicer arietinum
WNLTLYRYRNQGDLKATIL

SEQ ID NO: 56 - DNA, 15 bp, Arabidopsis thaliana
cattaccgtggacag

SEQ ID NO: 57 - DNA, 15 bp, Arabidopsis thaliana
cattaccatggacag

SEQ ID NO: 58 - DNA, 2277 bp, Arabidopsis thaliana
tctagaactagtggatcccccgggctgcaggaattcggcacgaggacggcgcaatcctctctccgtagccacattcaccgtccatcaaaaccagtggtcggattcactcacttctcctcccgttctcggatcgcagtggcggttctgtcccgagatgaaacatctatgactccaccgcctccaaagcttcctttaccacgtcttaaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactgagtttgaagctgtggtggataatccaaatctcaggaagaagaagaacccatgagagtcttcctcgtgatcatggttgttcttgagattctttagtctgttttctctcgggtctgtgtctgtcggatgagcgttttagccactgtagttcaatgagtaacctctatttgctacgaactctcattcctagatcgtgggttaccttttggtttctccaagcaatttgaggctagcctccaataaaaaatagtatttctagtatttgaaaaaacgctactttcgtggtatagagaaagataaagagagagagagagagagagagagagagagagagagagagagagagagagagagagagatgctcttgatattgctcttgatacaactctattattattgctcttaatccataatgaaagtgctttatgaaaaaaaaaaaaaaaaaaaaaaaaaaaaaaactcgagggggggcccggt

SEQ ID NO: 59 - protein, 751 aa, Arabidopsis thaliana
SRTSGSPGLQEFGTRTAQSSLRSHIHRPSKPVVGFTHFSSRSRIAVAVLSRDETSMTPPPPKLPLPRLKVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLCYLMGGRSTVGDEVLCRFTFPERPGALMNFLDSFSPRWNITLFHYHGQGETGANVLVGIQVPEQEMEEFKNRAKALGYDYFLVSDDDYFKLLMHVSCGGSKSQEEEEPMRVFLVIMVVLEILSVFSRVCVCRMSVLATVVQVTSICYELSFLDRGLPFGFSKQFEASLQKIVFLVFEKTLLSWYRERRERERERERERERERERERDALDIALDTTLLLLLLIHNESALKKKKKKKKKKLEGGPG

SEQ ID NO: 60 - protein, 712 aa, Arabidopsis thaliana
LELVDPPGCRNSARGRRNPLSVATFTVHQNQWSDSLTSPPVLGSQWRFCPEMKHLLHRLQSFLYHVLRSLRIRCNTLPVTSVLYQNVRTRLRTEASRKLWSIRIYCPLRFTTSPLSHHSNWLRSYLRDVFVCILKEKTCNLYSRLSFVELTIWNFQQINWQKELSALQLETMLKELLYLLVNSAALLLLCLLRLLRSGKLRIWVQRLFFSEIRMIKHKHMLRYELKKRVRLYLLLITLMLLLDKGLLGWRSLVRLRVHCMLYLCQLVVVVLVLLLMRGFLPRRSLVNQLTQMQWLCRCITVRGYWTRLGDLQMVQLKKLVKRLFVAEIWMVLFLSLVMLFVHQRICLRRNGTYWNQQGLLHSLELRHTVNIMARTMSPPVALTTLTSGLQNSPMSVGNRKLFLLLSCRKNLEALSNFVSWLDQTASSNIDVARKRRLLYYTVSEFTQLESSKHYRREWNLLNSKLSISLPVTKITCVTWEEDLLLETRFYADSPFPRDLVLTSWTLSVHGGTSPFSITMDRVRRARMCWSGSKSPSKKWRNLKTELKLLDTTTSVMTTILSFCTEFEAVVDNPNLRKKKNPESSSSWLFLRFFSLFSLGSVSVGAFPLFNEPLFATNSHSIVGYLLVSPSNLRLASNKKYFYLKKRYFRGIEKDKEREREREREREREREREREMLLILLLIQLYYYCSSIMKVLYEKKKKKKKKKNSRGGP

SEQ ID NO: 61 - protein, 706 aa, Arabidopsis thaliana
NWIPRAAGIRHEDGAILSPPHSPSIKTSGRIHSLLLPFSDRSGGSVPRNIYDSTASKASFTTSGLSEFVAIPCRLPRCCTRTYERGERKHRGSYGVFDEYTVHGLRHRHVTTPIGEAIEIRCSYVSKRRLATCILVASWSLQYDGETSSRSIGKRSYLLFSWKPCSRSCFICTRLHCCDCYACYDSDKVASCREFGCNGCSFRRFVSSTSTCDTSRRGSDVYTSFSPCYCWTRDCWDGDHSSGGSIACYICASWWWWFNSWYCCLCEEGFSRGEDHWCRTSRKCNGFVAASREGDIGPGWGICRWCSSRSWRDFSYKQKSNGWCCSCHSCYLCINKGYVGETEHIGTSRGSCTRWSGILILWPKGRECRSHNQWRHELQAKDCDRTRQCRATGSCSCYSHAGKTWKLAILAGWTNEHKRVQIMLGKGGCCTIQCRSSHSWRAQSTTEENGIFSTQNCQSHYQLSERSPVLLDGRKIYCWRRGSMPIHLSRETWCSNELLGLFQSTVEHHPFPLPWTGDGRECAGRDPSPRARNGGIKPSSSWIRLLLSKRLFASDALSLKLWWIIQISGRRRTHESLPRDHGCSDSLVCFLSGLCLSDERFSHCSSMSNLYLLRTLIPRSWVTFWFLQAIGPPIKNSISSIKNATFVVRKIKRERERERERERERERERERCSYCSYNSIIIALNPKCFMKKKKKKKKKKTRGGAR

SEQ ID NO: 62 - DNA, 2304 bp, Arabidopsis thaliana
  CDS (1)..(1827)
atgggcgagctcggtacccggggatcctctagaactagtggatcccccgggctgcaggaattcggcacgaggacggcgcaatcctctctccgtagccacattcaccgtccatcaaaaccagtggtcggattcactcacttctcctcccgttctcggatcgcagtggcggttctgtcccgagatgaaacatctatgactccaccgcctccaaagcttcctttaccacgtcttaaggtctctccgaattcgttgcaataccctgccggttacctcggtgctgtaccagaacgtacgaacgaggctgagaacggaagcatcgcggaagctatggagtatttgacgaatatactgtccactaaggtttacgacatcgccattgagtcaccactccaattggctaagaagctatctaagagattaggtgttcgtatgtatcttaaaagagaagacttgcaacctgtattctcgtttaagcttcgtggagcttacaatatgatggtgaaacttccagcagatcaattggcaaaaggagttatctgctcttcagctggaaaccatgctcaaggagttgctttatctgctagtaaactcggctgcactgctgtgattgttatgcctgttacgactcctgagataaagtggcaagctgtagagaatttgggtgcaacggttgttcttttcggagattcgtatgatcaagcacaagcacatgctaagatacgagctgaagaagagggtctgacgtttatacctccttttgatcaccctgatgttattgctggacaagggactgttgggatggagatcactcgtcaggctaagggtccattgcatgctatatttgtgccagttggtggtggtggtttaatagctggtattgctgcttatgtgaagagggtttctcccgaggtgaagatcattggtgtagaaccagctgacgcaaatgcaatggctttgtcgctgcatcacggtgagagggtgatattggaccaggttgggggatttgcagatggtgtagcagttaaagaagttggtgaagagacttttcgtataagcagaaatctaatggatggtgttgttcttgtcactcgtgatgctatttgtgcatcaataaaggatatgtttgaggagaaacggaacatattggaaccagcaggggctcttgcactcgctggagctgaggcatactgtaaatattatggcctaaaggacgtgaatgtcgtagccataaccagtggcgctaacatgaactttgacaagctaaggattgtgacagaactcgccaatgtcggtaggcaacaggaagctgttcttgctactctcatgccggaaaaacctggaagctttaagcaattttgtgagctggttggaccaatgaacataagcgagttcaaatatagatgtagctcggaaaaggaggctgttgtactatacagtgtcggagttcacacagctggagagctcaaagcactacagaagagaatggaatcttctcaactcaaaactgtcaatctcactaccagtgacttagtgaaagatcacctgtgttacttgatgggaggaagatctactgttggagacgaggttctatgccgattcacctttcccgagagacctggtgctctaatgaacttcttggactctttcagtccacggtggaacatcacccttttccattaccatggacagggtgagacgggcgcgaatgtgctggtcgggatccaagtccccgagcaagaaatggaggaatttaaaaaccgagctaaagctcttggatacgactacttcttagtaagtgatgacgactattttaagcttctgatgcactgagtttgaagctgtggtggataatccaaatctcaggaagaagaagaacccatgagagtcttcctcgtgatcatggttgttcttgagattctttagtctgttttctctcgggtctgtgtctgtcggatgagcgttttagccactgtagttcaatgagtaacctctatttgctacgaactctcattcctagatcgtgggttaccttttggtttctccaagcaatttgaggctagcctccaataaaaaatagtatttctagtatttgaaaaaacgctactttcgtggtatagagaaagataaagagagagagagagagagagagagagagagagagagagagagagagagagagagagagatgctcttgatattgctcttgatacaactctattattattgctcttaatccataatgaaagtgctttatgaaaaaaaaaaaaaaaaaaaaaaaaaaaaaaactcgagggggggcccggt

SEQ ID NO: 63 - protein, 609 aa, Arabidopsis thaliana; identical to SEQ ID NO: 6

SEQ ID NO: 64 - protein, 592 aa, Arabidopsis thaliana; identical to SEQ ID NO: 4

SEQ ID NO: 65 - protein, 555 aa, Arabidopsis thaliana
IPFSFRRRNPLSVATFTVHQNQWSDSLTSPPVLGSQWRFCPEMKHLLHRLQSFLYHVLRSLRIRCNTLPVTSVLYQNVRTRLRTEASRKLWSIRIYCPLRFTTSPLSHHSNWLRSYLRDVFVCILKEKTCNLYSRLSFVELTIWNFQQINWQKELSALQLETMLKELLYLLVNSAALLLLCLLRLLRSGKLRIWVQRLFFSEIRMIKHKHMLRYELKKRVRLYLLLITLMLLLDKGLLGWRSLVRLRVHCMLYLCQLVVVVLVLLLMRGFLPRRSLVNQLTQMQWLCRCITVRGYWTRLGDLQMVQLKKLVKRLFVAEIWMVLFLSLVMLFVHQRICLRRNGTYWNQQGLLHSLELRHTVNIMARTMSPPVALTTLTSGLQNSPMSVGNRKLFLLLSCRKNLEALSNFVSWLDQTASSNIDVARKRRLLYYTVSEFTQLESSKHYRREWNLLNSKLSISLPVTKITCVTWEEDLLLETRFYADSPFPRDLVLTSWTLSVHGGTSPFSITMDRVRRARMCWSGSKSPSKKWRNLKTELKLLDTTTSVMTTILSFCT

SEQ ID NO: 66 - protein, 551 aa, Arabidopsis thaliana
EFRSASDGAILSPPHSPSIKTSGRIHSLLLPFSDRSGGSVPRNIYDSTASKASFTTSGLSEFVAIPCRLPRCCTRTYERGERKHRGSYGVFDEYTVHGLRHRHVTTPIGEAIEIRCSYVSKRRLATCILVASWSLQYDGETSSRSIGKRSYLLFSWKPCSRSCFICTRLHCCDCYACYDSDKVASCREFGCNGCSFRRFVSSTSTCDTSRRGSDVYTSFSPCYCWTRDCWDGDHSSGGSIACYICASWWWWFNSWYCCLCEEGFSRGEDHWCRTSRKCNGFVAASREGDIGPGWGICRWCSSRSWRDFSYKQKSNGWCCSCHSCYLCINKGYVGETEHIGTSRGSCTRWSGILILWPKGRECRSHNQWRHELQAKDCDRTRQCRATGSCSCYSHAGKTWKLAILAGWTNEHKRVQIMLGKGGCCTIQCRSSHSWRAQSTTEENGIFSTQNCQSHYQLSERSPVLLDGRKIYCWRRGSMPIHLSRETWCSNELLGLFQSTVEHHPFPLPWTGDGRECAGRDPSPRARNGGIKPSSSWIRLLLSKRLFASDAL

SEQ ID NO: 67 - protein, 592 aa, Arabidopsis thaliana; identical to SEQ ID NO: 2

SEQ ID NO: 68 - protein, 555 aa, Arabidopsis thaliana
IPFSFRRRNPLSVATFTVHQNQWSDSLTSPPVLGSQWRFCPEMKHLLHRLQSFLYHVLRSLRIRCNTLPVTSVLYQNVRTRLRTEASRKLWSIRIYCPLRFTTSPLSHHSNWLRSYLRDVFVCILKEKTCNLYSRLSFVELTIWNFQQINWQKELSALQLETMLKELLYLLVNSAALLLLCLLRLLRSGKLRIWVQRLFFSEIRMIKHKHMLRYELKKRVRLYLLLITLMLLLDKGLLGWRSLVRLRVHCMLYLCQLVVVVLVLLLMRGFLPRRSLVNQLTQMQWLCRCITVRGYWTRLGDLQMVQLKKLVKRLFVAEIWMVLFLSLVMLFVHQRICLRRNGTYWNQQGLLHSLELRHTVNIMARTMSPPVALTTLTSGLQNSPMSVGNRKLFLLLSCRKNLEALSNFVSWLDQTASSNIDVARKRRLLYYTVSEFTQLESSKHYRREWNLLNSKLSISLPVTKITCVTWEEDLLLETRFYADSPFPRDLVLTSWTLSVHGGTSPFSITVDRVRRARMCWSGSKSPSKKWRNLKTELKLLDTTTSVMTTILSFCT

SEQ ID NO: 69 - protein, 551 aa, Arabidopsis thaliana
EFRSASDGAILSPPHSPSIKTSGRIHSLLLPFSDRSGGSVPRNIYDSTASKASFTTSGLSEFVAIPCRLPRCCTRTYERGERKHRGSYGVFDEYTVHGLRHRHVTTPIGEAIEIRCSYVSKRRLATCILVASWSLQYDGETSSRSIGKRSYLLFSWKPCSRSCFICTRLHCCDCYACYDSDKVASCREFGCNGCSFRRFVSSTSTCDTSRRGSDVYTSFSPCYCWTRDCWDGDHSSGGSIACYICASWWWWFNSWYCCLCEEGFSRGEDHWCRTSRKCNGFVAASREGDIGPGWGICRWCSSRSWRDFSYKQKSNGWCCSCHSCYLCINKGYVGETEHIGTSRGSCTRWSGILILWPKGRECRSHNQWRHELQAKDCDRTRQCRATGSCSCYSHAGKTWKLAILAGWTNEHKRVQIMLGKGGCCTIQCRSSHSWRAQSTTEENGIFSTQNCQSHYQLSERSPALLDGRKIYCWRRGSMPIHLSRETWCSNELLGLFQSTVEHHPFPLPWTGDGRECAGRDPSPRARNGGIKPSSSWIRLLLSKRLFASDAL

(19) United States
(12) Patent Application Publication
(10) Pub. No.: US 2004/0006784 A1
(43) Pub. Date: Jan. 8, 2004

(54) Methods and compositions for producing plants and microorganisms that express feedback insensitive threonine dehydratase/deaminase

(76) Inventor: George S. Mourad, Fort Wayne, IN (US)

Correspondence Address: Jaen Andrews, MEDLEN & CARROLL, LLP, 101 Howard Street, Suite 350, San Francisco, CA, 94105, US

(21) Appl. No.: 10/413,943

(22) Filed: Apr. 15, 2003

Related U.S. Application Data
(63) Continuation of application No. 09/226,955, filed on Jan. 8, 1999, now abandoned.
Application No. 09/226,955 is a continuation of application No. PCT/US98/14362, filed on Jul. 10, 1998.
(60) Provisional application No. 60/074,875, filed on Feb. 17, 1998.
(60) Provisional application No. 60/052,096, filed on Jul. 10, 1997.

(51) Int. Cl. A01H 1/00; C12N 15/82; C12N 9/10; C07H 21/04; C12N 5/04

(52) U.S. Cl. 800/278; 435/69.1; 435/320.1; 435/419; 530/370; 536/23.6; 435/193

(57) ABSTRACT

The present invention relates to methods and materials in the field of molecular biology and the regulation of polypeptide synthesis through genetic engineering of plants and/or microorganisms. More particularly, the invention relates to newly-isolated nucleotide sequences, nucleotide sequences having substantial identity thereto and equivalents thereof, as well as polypeptides encoded thereby. The invention also involves the introduction of foreign nucleotide sequences into the genome of a plant and/or microorganism, wherein the introduction of the nucleotide sequence effects an increase in the transformant's resistance to toxic isoleucine structural analogs. Inventive sequences may therefore be used as excellent molecular markers for screening successful transformants, thereby replacing antibiotic resistance genes used in the prior art. Transformants harboring a nucleotide sequence comprising a promoter operably linked to an inventive nucleotide sequence demonstrate increased levels of isoleucine production, thereby providing an improved nutrient source.

### REFERENCES TO RELATED APPLICATIONS

This application claims the benefit of U.S. Provisional Application No. 60/052,096, filed Jul. 10, 1997 and entitled cDNA CLONE SEQUENCE OF THREONINE DEHYDRATASE/DEAMINASE FROM ARABIDOPSIS THALIANA; and U.S. Provisional Application No. 60/074,875, filed Feb. 17, 1998 and entitled THE MOLECULAR BASIS OF L-O-METHYLTHREONINE RESISTANCE ENCODED BY THE omr1 ALLELE OF LINE GM11b OF ARABIDOPSIS THALIANA; both of which are hereby incorporated by reference herein in their entirety.

### BACKGROUND OF THE INVENTION

1. Field of the Invention

The present invention relates to methods and materials in the field of molecular biology and to the utilization of isolated nucleotide sequences to genetically engineer plants, and/or microorganisms. More particularly, the invention relates in certain preferred aspects to novel nucleotide sequences and uses thereof, including their use in DNA constructs for transforming plants, fungi, yeast & bacteria. The nucleotide sequences are particularly useful as selectable markers for screening plants and/or microorganisms for successful transformants and also for improving the nutritional value of plants.

2. Introduction and Discussion of Related Art

Threonine dehydratase/deaminase (“TD”) is the first enzyme in the biosynthetic pathway of isoleucine, and catalyzes the formation of 2-oxobutyrate from threonine (“Thr”) in a two-step reaction. The first step is a dehydration of Thr, followed by rehydration and liberation of ammonia. All reactions downstream from TD are catalyzed by enzymes that are shared by the two main branches of the biosynthetic pathway that lead to the production of the branched-chain amino acids, isoleucine (“Ile”), leucine (“Leu”), and valine (“Val”). An illustration of the biosynthetic pathway is set forth in FIG. 1. The cellular levels of Ile are controlled by negative feedback inhibition. When the cellular levels of Ile are high, Ile binds to TD at a regulatory site (allosteric site) that is different from the substrate binding site (catalytic site) of the enzyme. The formation of this Ile-TD complex causes conformational changes to TD, which prevent the binding of substrate, thus inhibiting the Ile biosynthetic pathway.

It is known that certain structural analogs of Ile exist which are toxic to a wide variety of plants and microorganisms. It is believed that these Ile analogs are toxic because cells incorporate the analogs into polypeptides in place of Ile, thereby synthesizing defective polypeptides. In this regard, L-O-methylthreonine (“OMT”) was reported in 1955 to be a structural analog of Ile that inhibits growth of manmalian cell cultures by inhibiting incorporation of Ile into proteins. (Rabinovitz M, et al., Steric relationship between threonine and isoleucine as indicated by an antimetabolite study. J Am Chem Soc 77:3109-3111 (1955).) It is believed that the same phenomenon explains growth inhibition, which is caused by other structural analogs of Ile such as, for example, thiaIle.

Certain strains of bacteria and yeast and certain plant lines have been identified which are resistant to the toxicity of the above-noted Ile structural analogs, and this resistance has been attributed to a mutation in the TD enzyme. The mutated TD apparently features a loss or decrease of Ile feedback sensitivity (referred to herein as “insensitivity”). As a result of this insensitivity, cells harboring insensitive TD produce increased amounts of Ile, thereby outcompeting the toxic Ile analog during incorporation into cellular proteins. For example, resistance to thiaIle has been associated in certain strains of bacteria and yeast with a loss of feedback sensitivity of TD to Ile. In Rosa cells, resistance to OMT was also associated with a TD that had reduced sensitivity to feedback inhibition by Ile. Being in tissue culture and having high ploidy level, however, it was not possible to determine the genetic basis of feedback insensitivity to Ile in the Rosa variant, the only known plant mutated with an Ile-insensitive TD.

Turning to a field of research where the present invention finds advantageous application, selectable markers are widely used in methods for genetically transforming cells, tissues and organisms. Such markers are used to screen cells, most commonly bacteria, to determine whether a transformation procedure has been successful. As a specific example, it is widely known that constructs for transforming a cell may include as a selectable marker a nucleotide sequence that confers antibiotic resistance to the transformed cell. As used herein in connection with cells and plants, the terms “transformed” and “transgenic” are used interchangeably to refer to a cell or plant expressing a foreign nucleotide sequence introduced through transformation efforts. The term “foreign nucleotide sequence” is intended to indicate a sequence encoding a polypeptide whose exact amino acid sequence is not normally found in the host cell, but is introduced therein through transformation techniques. After transformation, the cells may be contacted with an antibiotic in a screening procedure. Only successful transformants, i.e., those which possess the antibiotic resistance gene, survive and continue to grow and proliferate in the presence of the antibiotic. This techniques provides a manner whereby successful transformants may be identified and propagated, thereby eliminating the time consuming and costly alternative of growing and working with cells which were not successfully transformed.

The above-described screening technique is becoming less advantageous, however, because, due to prolonged exposure to antibiotics, an ever-increasing number of naturally-occurring microorganisms are developing antibiotic resistance by spontaneous mutation. The reliability of this screening technique is therefore compromised because the continuous exposure to antibiotics causes microorganisms that are not transformed to develop spontaneo'us mutations that confer antibiotic resistance.

In addition to the decreasing viability of this screening technique, the overuse of antibiotics, and the resulting resistance spontaneously developed by microorganisms, is a growing medical concern as the efficacy of antibiotics in fighting bacterial infections is decreasing. Many infections—including meningitis—no longer respond well to drugs that once worked well against them. This phenomenon is attributed largely to the overuse of antibiotics, both as drugs and as a laboratory screening tool, and the resulting antibiotic resistance of a growing number of microorganisms. As an example, the bacteria that causes meningitis once was routinely controlled with ampicillin, a commonly prescribed antibiotic and an antibiotic very heavily used in screening transformed bacterial cells for resistance as a selectable marker. Now, however, about 20 percent of such infections are resistant to ampicillin.

The present invention addresses the aforementioned problems in screening genetic transformants and provides nucleotide sequences which may be advantageously used as selectable markers, and which may be inserted into the genome of a plant or microorganism to provide a transformed plant or microorganism. Such a transformed plant or microorganism advantageously exhibits significantly increased levels of Ile synthesis and synthesis of intermediates of the Ile biosynthetic pathway and is therefore also capable of surviving in the presence of a toxic Ile analog.

### SUMMARY OF THE INVENTION

The present invention provides nucleotide sequences, originally isolated and cloned from Arabidopsis thaliana, which encode feedback insensitive TD that may advantageously be used to transform a wide variety of plants, fungi, bacteria and yeast. Inventive forms of TD are not only insensitive to feedback inhibition by isoleucine, but are also insensitive to structural analogs of isoleucine that are toxic to plants and microorganisms which synthesize only wild-type TD. Therefore, inventive nucleotide sequences encoding mutated forms of TD can be used to create cells that are insensitive to compounds normally toxic to cells expressing only wild-type TD enzymes. In this regard, an inventive nucleotide sequence may be used in a DNA construct to provide a biochemical selectable marker

One aspect of the present invention is identification, isolation and purification of a gene encoding a wild-type form of TD. The DNA sequence thereof can be used as disclosed herein to determine the complete amino acid sequence for the protein encoded thereby and thus allow identification of domains found therein that can be mutated to produce additional TD proteins having altered enzymatic characteristics. In another aspect of the invention, there are provided isolated and purified polynucleotides, the polynucleotides encoding a mutated form of TD, or a portion thereof, as disclosed herein. For example, the invention provides isolated polynucleotides comprising the sequence set forth in SEQ ID NO:2, SEQ ID NO:3 and SEQ ID NO:4, nucleotide sequences having substantial identity thereto, and nucleotide sequences encoding TD variants of the invention. Also provided are isolated polypeptides comprising the amino acid sequence set forth in SEQ ID NO:2, SEQ ID NO:3 and SEQ ID NO:4 and variants thereof selected in accordance with the invention.

In an alternate aspect of the invention, there is provided a chimeric DNA construct comprising a promoter operably linked to a nucleotide sequence encoding a threonine dehydratase/deaminase that is substantially resistant to feedback inhibition. In a cell harboring the construct, the nucleotide sequence can be transcribed to produce mRNA and said mRNA can be translated to produce either mature, mutated TD or a precursor mutated TD protein, said protein being functional in said cell. Also provided, therefore, is a vector useful for transforming a cell, and plants and microorganisms transformed therewith, the vector comprising a DNA construct selected in accordance with the invention. In alternate aspects of the invention, there are provided cells and plants having incorporated into their genome a foreign nucleotide sequence operably linked to a promoter, the foreign sequence comprising a nucleotide sequence having substantial identity to a sequence set forth herein or a foreign nucleotide sequence encoding an inventive polypeptide.

In another aspect of the invention, there is provided a method comprising incorporating into a plant's genome an inventive DNA construct to provide a transformed plant; wherein the transformed plant is capable of expressing the nucleotide sequence.

Yet another aspect of the invention is the production and propagation of cells transformed in accordance with the invention, wherein the cells express a mutated TD enzyme, thus making the cells resistant to feedback inhibition by isoleucine, and resistant to molecules that are toxic to a cell producing only the wild-type TD enzyme. In this regard, there is provided a method comprising providing a vector featuring a promoter operably linked to a nucleotide sequence encoding a threonine dehydratase/deaminase that is resistant to feedback inhibition, wherein the promoter regulates expression of the nucleotide sequence in a host plant cell; and transforming a target plant with the vector to provide a transformed plant, the transformed plant being capable of expressing the nucleotide sequence. Plants transformed in accordance with the invention have within their chloroplasts a mature, mutated form of TD, which renders the cells resistant to toxic Ile analogs. Also provided are transformed plants obtained according to inventive methods and progeny thereof.

Also provided is a method for screening potential transformants, comprising (1) providing a plurality of cells, wherein at least one of the cells has in its genome an expressible foreign nucleotide sequence selected in accordance with the invention; and (2) contacting the plurality of cells with a substrate comprising a toxic isoleucine structural analog; wherein cells comprising the, expressible foreign nucleotide sequence are capable of growing in the substrate, and wherein cells not comprising the expressible foreign nucleotide sequence are incapable of growing in the substrate.

In another aspect of the invention, there is provided a construct comprising a primary nucleotide sequence to be introduced into the genome of a target cell, tissue and/or organism, and further comprising a biochemical selectable marker selected in accordance with the invention. This aspect of the invention may be advantageously used to transform a wide variety of cells, including microorganisms and plant cells. After introducing the DNA construct, which also includes an appropriate promoter and such other regulatory sequences as may be selected by a skilled artisan, into a target plant or microorganism, the plant or microorganism may be grown in a substrate comprising a toxic isoleucine analog (a “toxic substrate”), thereby providing a mechanism for the early determination whether the transformation was successful. Where a plurality of plants or microorganisms are transformed, placing potential transformants into a toxic substrate provides an early screening step whereby successful transformants may be identified. It is readily understood by a person skilled in the relevant field, in view of the present specification, that successful transformants will grow normally in the toxic substrate by virtue of expression of the insensitive TD; however, unsuccessfully transformed plants and/or microorganisms will die due to the toxic effect of the substrate. Transformed plants may thereby be identified quickly in accordance with the invention, and transformed microorganisms may be identified in accordance with the invention without using antibiotic resistance genes.

In another aspect of the invention, there is provided a method for reliably incorporating a first, expressible, foreign nucleotide sequence into a target cell, comprising providing a vector comprising a promoter operably linked to a first primary nucleotide sequence and a second nucleotide sequence selected in accordance with the invention, the second sequence encoding an insensitive TD enzyme; transforming the target cell with the vector to provide a transformed cell; and contacting the cell with a substrate comprising L-O-methylthreonine; wherein successfully transformed cells are capable of growing in the substrate, and wherein unsuccessfully transformed cells are incapable of growing in the substrate.

In an alternate aspect of the invention, there is provided a method for growing a plurality of plants in the absence of undesirable plants, such as, for example, weeds, the method comprising providing a plurality of plants, each having in its genome a foreign nucleotide sequence comprising a promoter operably linked to a nucleotide sequence selected in accordance with the invention; growing the plurality of plants in a substrate; and introducing a preselected amount of an isoleucine structural analog into the substrate.

TD enzymes described herein function in the chloroplasts of a plant cell. Therefore, it is readily appreciated by a skilled artisan that a nucleotide sequence inserted into a plant cell will necessarily encode a precursor TD peptide. Thus, chimeric DNA constructs are described herein that comprise a first nucleotide sequence encoding a mature mutated form of TD and a second nucleotide sequence encoding a chloroplast transit peptide of choice, the second sequence being functionally attached to the 5′ end of the first sequence. Expression of the chimeric DNA construct results in the production of a mutated precursor TD enzyme that can be translocated to a chloroplast. The presence of a mature mutated TD in the chloroplast results in a plant cell having characteristics described herein.

It is an object of the present invention to provide isolated nucleotide sequences, which may be introduced into the genome of a plant or microorganism to increase the ability of the plant or microorganism to synthesize Ile and intermediates of the Ile biosynthetic pathway.

Additionally, it is an object of the invention to provide nucleotide sequences, which may be used as excellent biochemical selectable markers for identifying successful transformants in genetic engineering protocols.

It is also an object of the invention to provide a novel, efficient, selective, environmentally-friendly herbicide system.

Further objects, advantages and features of the present invention will be apparent from the detailed description herein.

### BRIEF DESCRIPTION OF THE FIGURES

Although the characteristic features of this invention will be particularly pointed out in the claims, the invention itself, and the manner in which it may be made and used, may be better understood by referring to the following description taken in connection with the accompanying figures forming a part hereof.

FIG. 1 illustrates the biosynthetic pathway of the branched-chain amino acids valine, leucine and isoleucine.

FIG. 2 sets forth the alignment of the amino acid sequence of TD of tomato and chickpea. C regions are highly conserved regions of the catalytic site of TD while R regions are highly conserved regions of the regulatory site of TD. Also shown are the locations of the degenerate oligonucleotide primers TD205 and TD206 used to PCR-amplify an Arabidopsis TD genomic DNA fragment

FIG. 3 sets forth the structure and degree of degeneracy of the two oligonucleotide primers TD205 and TD206 used in amplifying an Arabidopsis genomic DNA fragment of the TD gene omr1. TD205 is anchored with an Eco RI site (underlined) at its 5′ end and TD206 is anchored with a Hind III site (underlined) at its 5′ end.

FIG. 4 sets forth the DNA sequence of clone 23 (pGM-td23) isolated from a cDNA library of the mutated line GM11b (omr1/omr1) of Arabidopsis thaliana.

FIG. 5 sets forth the nucleotide sequence and the predicted amino acid sequence of clone 23 as isorated from the cDNA library constructed from line GM11b of Arabidopsis (omr1/omr1). The TD insert in clone 23 is in pBluescript vector between the Eco RI and Xho I sites. An open reading frame (top reading frame) was observed which showed an ATG codon at nucleotide 166 and a termination codon at nucleotide 1801.

FIG. 6a depicts the structure of the expression vector pCM35S-omr1 used in the transformation of wild-type Arabidopsis thaliana and which expressed a mutated form of TD capable of conferring resistance to the toxic analog L-O-methylthreonine upon transformants.

FIG. 6b sets forth the nucleotide sequence and the predicted amino acid sequence of the chimeric mutant omr1 expressing resistance to L-O-methylthreonine in transgenic Arabidopsis plants that have been transformed with the expression vector pCM35s-omr1 (shown in FIG. 6a). The total length of the fusion (chimeric) mutant TD expressed in transgenic plants was 609 amino acid residues. The first 9 amino-terminal residues start by methionine encoded by a start codon (ATG) furnished by the 3′ end of the nucleotide sequence of CaMV 35s promoter linked to the omr1 insert of clone 23. The following 15 amino acid residues are generated by the nucleotide sequence of the polylinker region from the multiple cloning site of the vector and finally the remaining 585 amino acid residues are encoded by the omr1 mutant allele of Arabidopsis as present in clone 23. The first residue of the 585 amino acid long portion encoded by omr1 in pCM35s-omr1 corresponds to threonine (Thr) which is the amino-terminal residue number 8 of the full length omr1 cDNA shown in FIGS. 8 and 9 and SEQ ID NO:2.

FIG. 7 is the nucleotide sequence of the full length cDNA of the omr1 allele encoding mutated TD. The total length of the cDNA of omr1 is 1779 nucleotides including the stop codon.

FIG. 8 is the predicted amino acid sequence of the mutated TD encoded by omr1. The total length of the TD protein encoded by omr1 is 592 amino acids.

FIG. 9 is the nucleotide sequence and the predicted amino acid sequence encoded by the mutated allele omr1 of line GM11b of Arabidopsis thaliana.

FIG. 10 is the nucleotide sequence of the full length cDNA of the wild type allele OMR1 encoding wild type TD.

FIG. 11 is the predicted amino acid sequence of the wild type TD encoded by OMR1.

FIG. 12 is the nucleotide sequence and the predicted amino acid sequence encoded by the wild type allele OMR1 of Arabidopsis thaliana Columbia wild type.

FIG. 13 sets forth the multi-alignment of the deduced amino acid sequence of the wild-type TD of Arabidopsis thaliana reported in this disclosure with that from other organisms obtained from GenBank with the following accession numbers: 940472 for chickpea; 10257 for tomato; 401179 for potato; 730940 for yeast 1; 134962 for yeast 2; 68318 for E. coli biosynthetic; 135723 for E. coli catabolic; 1174668 for Salmonella typhimurium. The megalign program of the Lasergene software, DNASTAR Inc., Madison, Wis. was used.

FIG. 14 is a portion of the DNA sequencing gel comparing the nucleotide sequence of the mutated omr1 allele and its wild-type allele OMR1 and showing the base substitution C (in OMR1) to T (in omr1) at nucleotide residue 1495 starting from the beginning of the coding sequence. The arrow is pointing to the base substitution.

FIG. 15 depicts the point mutation in omr1 at nucleotide residue 1495, predicting an amino acid substitution, from arginine (R) to cysteine (C) at amino acid residue 499 at the TD level.

FIG. 16 sets forth the amino acid sequence at the regulatory region R4 of TD encoded by mutated omr1 and wild type OMR1) alleles of Arabidopsis thaliana compared to that from several organisms. The arrow points to the mutated amino acid residue in omr1.

FIG. 17 is a portion of the DNA sequencing gel comparing the nucleotide sequence of the mutated omr1 allele and its wild-type allele OMR1 and showing the base substitution G (in OMR1) to A (in omr1) at nucleotide residue 1631. The arrow is pointing to the base substitution.

FIG. 18 depicts the point mutation in omr1 at nucleotide residue 1631, predicting an amino acid substitution, arginine (R) to histidine (H) at amino acid residue 544 at the TD level.

FIG. 19 sets forth the amino acid sequence at the regulatory region R6 of TD encoded by mutated omr1 and wild type OMR1 alleles of Arabidopsis thaliana compared to that from several organisms. The arrow points to the mutated amino acid residue in omr1.

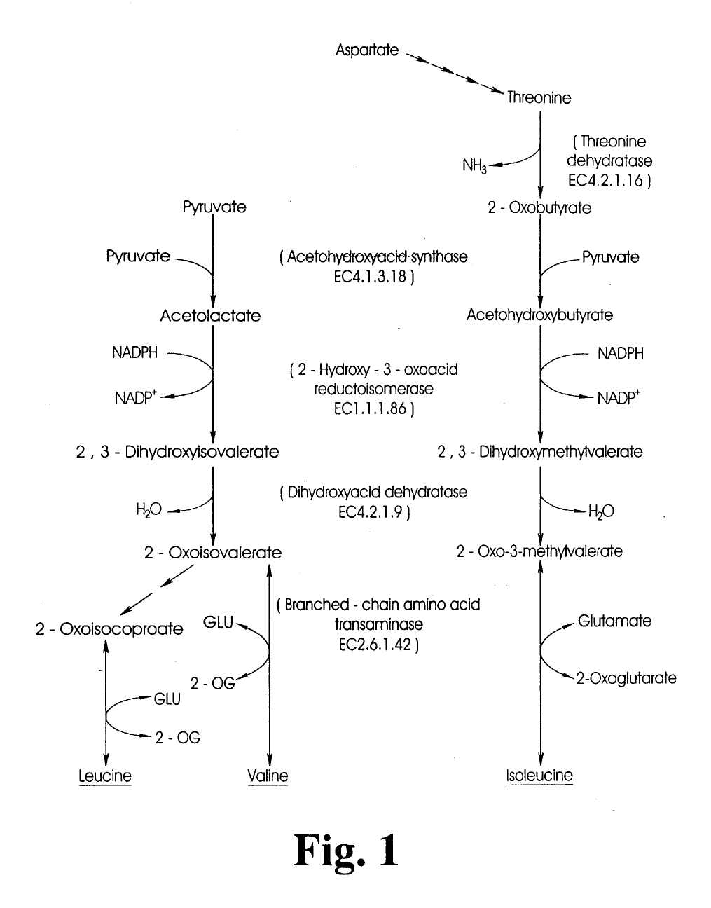

### DETAILED DESCRIPTION OF THE INVENTION

For purposes of promoting an understanding of the principles of the invention, reference will now be made to particular embodiments of the invention and specific language will be used to describe the same. It will nevertheless be understood that no limitation of the scope of the invention is thereby intended, such alterations and further modifications in the invention, and such further applications of the principles of the invention as described herein being contemplated as would normally occur to one skilled in the art to which the invention pertains.

As disclosed above, the present invention relates to methods and compositions for obtaining transformed cells, said cells expressing therein a mutated form of threonine dehydratase/deaminase (“TD”). More particularly, the invention provides isolated nucleotide sequences encoding mutated TD-functional polypeptides (“mutated TD”) which are resistant to Ile feedback inhibition and are resistant to the toxic effect of Ile analogs. These inventive nucleotide sequences can be incorporated into vectors, which in turn can be used to transform cells. Such transformation can be used, for instance, for purposes of providing a selectable marker, to increase plant nutritional value or to increase the production of commercially-important intermediates of the isoleucine biosynthetic pathway. Expression of the mutated TD results in the cell having altered susceptibility to certain enzyme inhibitors relative to cells having wild-type TD only. These and other features of the invention are described in further detail below.

One feature of the present invention involves the discovery, isolation and characterization of a gene sequence from Arabidopsis thaliana, designated omr1, which encodes a surprisingly advantageous mutated form of the enzyme TD. Aspects of the present invention thus relate to nucleotide sequences encoding mutated forms of TD, which sequences may be introduced into target plant cells or microorganisms to provide a transformed plant or microorganism having a number of desirable features. The mutated forms of TD, unlike wild-type TD, are resistant to negative feedback inhibition by isoleucine (“Ile”) and transformed cells are resistant to molecules which are toxic to cells that do not express feedback insensitive TD. Therefore, transformants harboring an expressible inventive nucleotide sequence demonstrate increased levels of isoleucene production and increased levels of production of intermediates in the Ile biosynthetic pathway, and the transformants are resistant to Ile structural analogs which are lethal to non-transformants, which express only wild-type TD.

The present invention relates in another aspect to amino acid sequences that comprise functional, feedback-insensitive TD enzymes. The term “amino acid sequence” is used herein to designate a plurality of amino acids linked in a serial array. Skilled artisans will recognize that through the process of mutation and/or evolution, polypeptides of different lengths and having differing constituents, e.g., with amino acid insertions, substitutions, deletions, and the like, may arise that are related to a sequence set forth herein by virtue of amino acid sequence homology and advantageous functionality as described in detail herein. The term “TD enzyme” is used to refer generally to a wild-type TD amino acid sequence, to a mutated TD selected in accordance with the invention, and to variants of each which catalyzes the reaction of threonine to 2-oxobutyrate in the Ile biosynthetic pathway, as described herein. For purposes of clarity, the wild-type form is distinguished from a mutated form, where necessary, by usage of the terms “wild-type TD” and “mutated TD.”

It is not intended that the present invention be limited to the specific sequences set forth herein. It is well known that plants and microorganisms of a wide variety of species commonly express and utilize analogous enzymes and/or polypeptides which have varying degrees of degeneracy, and yet which effectively provide the same or a similar function. For example, an amino acid sequence isolated from one species may differ to a certain degree from the wild-type sequence set forth in SEQ ID NO:1, and yet have similar functionality with respect to catalitic and regulatory function. Amino acid sequences comprising such variations are included within the scope of the present invention and are considered substantially similar to a reference amino acid sequence. It is believed that the identity between amino acid sequences that is necessary to maintain proper functionality is related to maintenance of the tertiary structure of the polypeptide such that specific interactive sequences will be properly located and will have the desired activity. While it is not intended that the present invention be limited by any theory by which it achieves its advantageous result, it is contemplated that a polypeptide including these interactive sequences in proper spatial context will have good activity, even where alterations exist in other portions thereof.

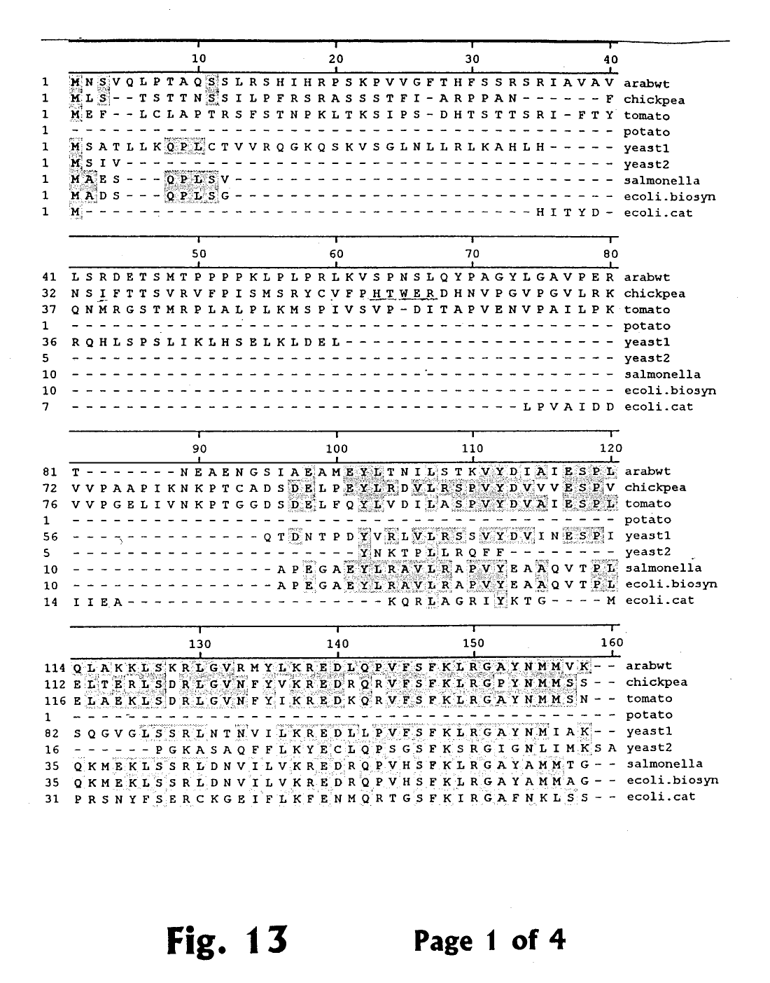

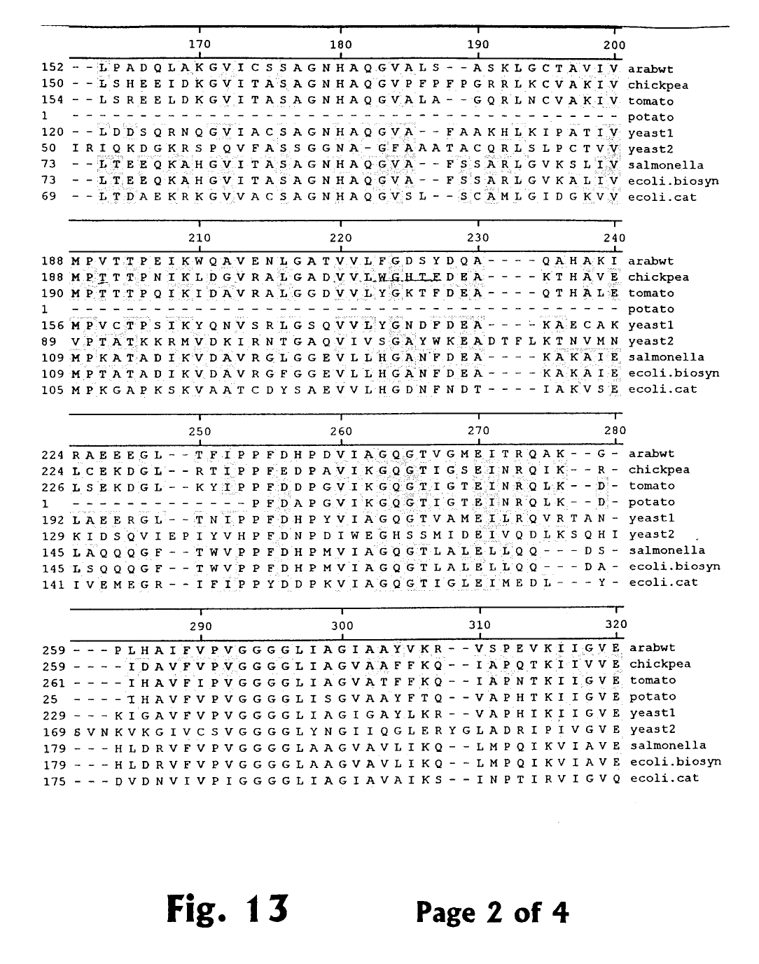

In this regard, a TD variant is expected to be functionally similar to the wild-type TD set forth in SEQ ID NO:1, for example, if it includes amino acids which are conserved among a variety of species or if it includes non-conserved amino acids which exist at a given location in another species that expresses functional TD. FIG. 13 sets forth an amino acid alignment of TD polypeptides of a number of species. Two significant observations which may be made based upon FIG. 13 are (1) that there is a high degree of conservation of amino acids at many locations among the species shown, and (2) a number of insertions, substitutions and/or deletions are represented in the TD of certain species and/or strains, which do not eliminate the dual functionality of the respective TD enzymes. For example, on Page 4 of FIG. 13, Regulatory Region 4 (“R4”) of wild-type Arabidopsis is depicted which comprises the following sequence (corresponding to the underlying three-letter codes numbered as set forth in SEQ ID NO:1):

V N L T T S D L V K D H L R Y L M G G
Val Asn Leu Thr Thr Ser Asp Leu Val Lys Asp His Leu Arg Tyr Leu Met Gly Gly
486 490 495 500

The degeneracy shown in FIG. 13 in this portion of the sequence provides examples of substitutions which may be made without substantially altering the functionality of the wild-type sequence set forth in SEQ ID NO:1. For example, it is expected that the Asp (“D”) at position 492 could be substituted with a Glu (“E”) and that the Leu (“L”) at position 493 could be substituted with a Met (“M”) without substantially altering the functionality of the amino acid sequence.

The following sets forth a plurality of sequences of R4, depicted such that acceptable substitutions are set forth at various amino acid locations. The sequences encompassed thereby are expected to exhibit similar functionality to the corresponding portion of SEQ ID NO:1. A slash (“/”) between two or in a series of amino acids indicates that any one of the amino acids indicated may be present at that location.

```
Val/Leu/Phe/Ile Asn/Asp/Glu/Ser Leu/ILe/Phe/Val/Gly Thr/Ser/Ala/Gly
486
Thr/His/Asp/Asn Ser/Asn/Asp/Ile Asp/Glu Leu/Met Val/Ala Lys/Val/Ala
490 495
Asp/Ile/Glu/Ser His Leu/Gly/Ile/Val Arg/Lys Tyr/His Leu/Met Met/Val
                                                                                        500
Gly Gly
        504
```

It is understood that analogous substitutions throughout the sequence are encompassed within the scope of the invention, and that Region R4 is simply used above for purposes of illustration.

Another manner in which similarity may exist between two amino acid sequences is where a given amino acid is substituted with another amino acid from the same amino acid group. In this manner, it is known that serine may commonly be substituted with threonine in a polypeptide without substantially altering the functionality of the polypeptide. The following sets forth groups of amino acids which are believed to be interchangeable in inventive amino acid sequences at a wide variety of locations without substantially altering the functionality thereof:

| | |
|---|---|
| Group I: | Nonpolar amino acids: Alanine, valine, proline, leucine, |
| | phenylalanine, tryptophan, methionine, isoleucine, cysteine, |
| | glycine; |
| Group II: | Uncharged polar amino acids: Serine, threonine, asparagine, |
| | glutamine, tyrosine; |
| Group III: | Charged polar acidic amino acids: Aspartic, glutamic; and |
| Group IV: | Charged polar basic amino acids: Lysine, arginine, histidine. |

Where one is unsure whether a given substitution will affect the functionality of the enzyme, this may be determined without undue experimentation using synthesis techniques and screening assays known in the art.

Having established the meaning of similarity with respect to an amino acid sequence, it is important to note that the invention features mutated amino acid sequences comprising one or more amino acid substitutions that do alter the functionality of the wild-type TD enzyme. Inventive insensitive TD enzymes are therefore not similar to wild-type TD, as that term is defined and used herein, because inhibition functionality is altered. Insensitive TD enzymes feature one or more mutations in the regulatory site which mutations alter the functionality of the regulatory site without substantially altering the functionality of the catalytic site. In one specific aspect of the invention, there is provided an amino acid sequence (SEQ ID NO:2) having two substitutions, this sequence comprising a mutated TD which has good catalytic functionality but which does not exhibit regulatory functionality. In other words, the enzyme set forth in SEQ ID NO:2 comprises a feedback insensitive Arabidopsis thaliana TD.

It is seen upon comparing the wild type TD set forth in SEQ ID NO:1 and the mutated sequence of SEQ ID NO:2, which comprises a specific embodiment of the invention, that the sequences differ only by two point mutations in the respective nucleotide sequences (C to T at nucleotide 1495; and G to A at nucleotide 1631), which result in two amino acid substitutions in the TD polypeptide (Arg to Cys at amino acid location 499; and Arg to His at amino acid location 544). The first mutation is in regulatory region R4 of TD, and the second is in regulatory region R6 of TD. The Arg to Cys substitution at amino acid residue 499 changed a charged, polar, basic amino acid (Arg) to a nonpolar amino acid (Cys) which altered the feedback site in TD. On the other hand, the change of Arg to His at residue 544 was a change from a charged, polar, basic amino acid (Arg) to another charged, polar, basic amino acid (His). While it is not intended that the present invention be limited by any theory by which it achieves its advantageous result, it is believed that the substitution at residue 544 alone may not have substantially altered the feedback site of TD, and, in contrast, that the substitution at residue 499 alone may have desensitized TD encoded thereby to feedback regulation. Certainly, when combined, the substitutions were very effective in desensitizing TD encoded by omr1 to feedback regulation.

It is recognized that the amino acid sequence set forth in SEQ ID NO:3 (585 residues encoded by omr1) is a truncated version, missing 7 amino-terminal residues, of that set forth in SEQ ID NO:2. It is seen from the following description, including the Examples set forth herein, that a significant amount of research was performed based upon this slightly shortened version, and that the slightly shortened version may be advantageously used to transform a wide variety of plants and microorganisms. It is believed that the portion of the amino acid sequence that is present in SEQ ID NO:2 and absent in SEQ ID NO:3 is a portion of the chloroplast leader sequence, and not present in the mature TD enzyme.

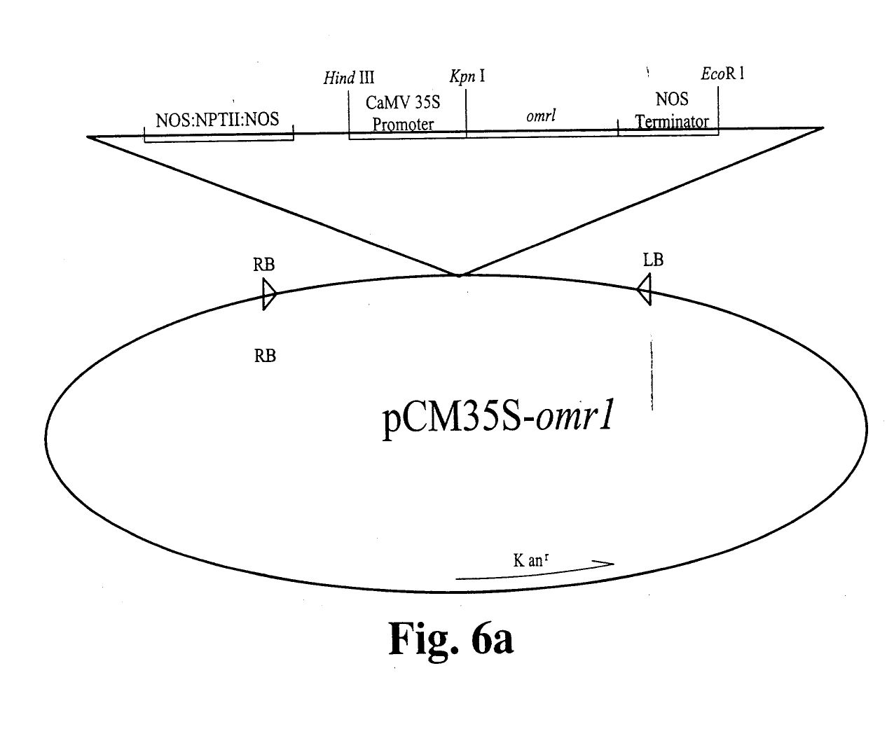

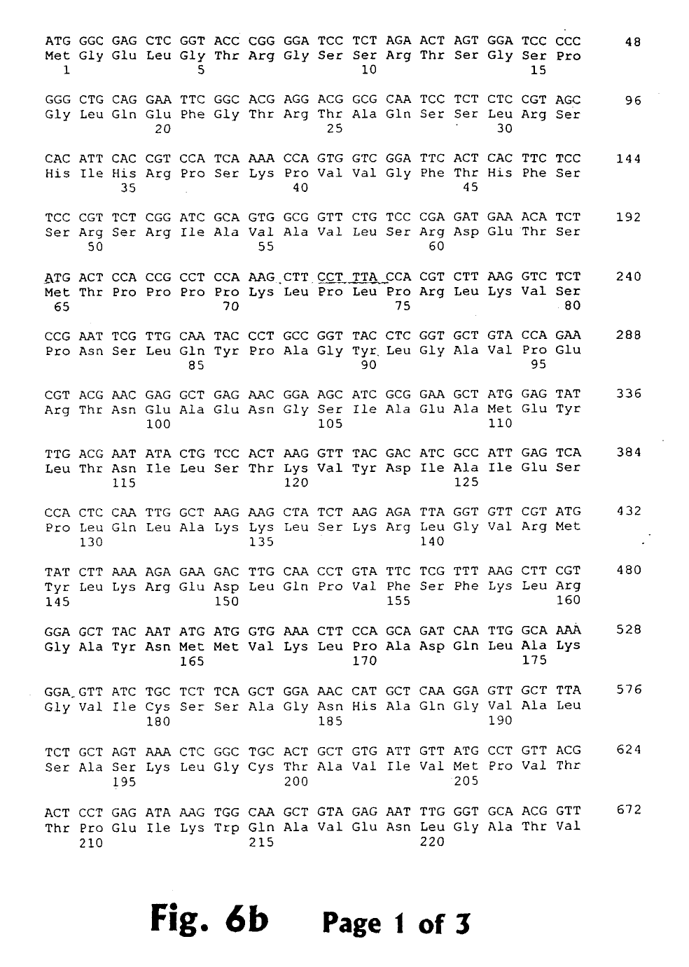

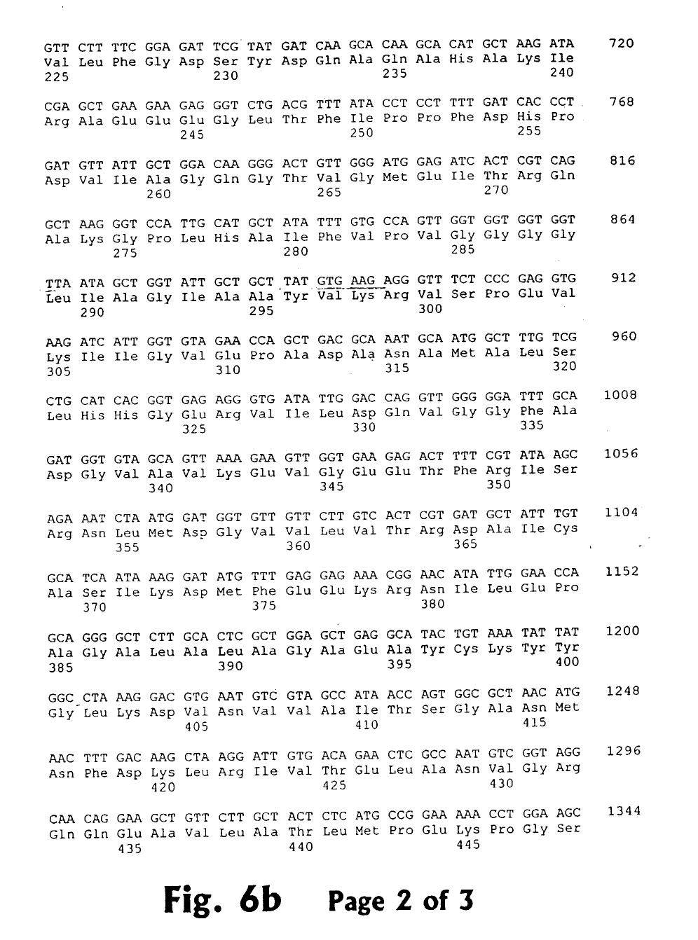

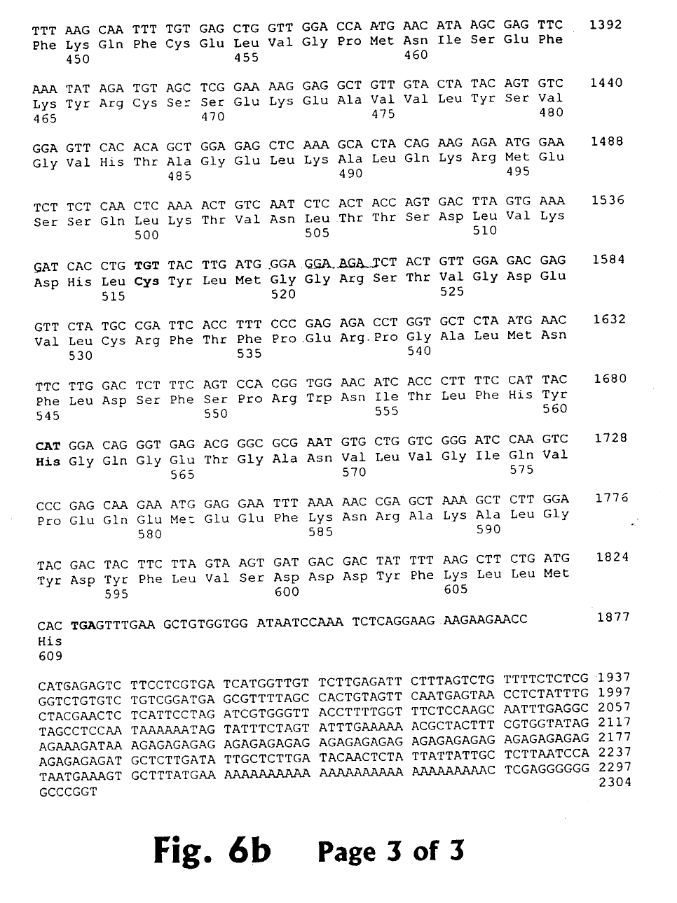

As mentioned above, to assist in the description of the present invention, SEQ ID NO:1 is provided which sets forth a nucleotide sequence, and the amino acid sequence encoded thereby, comprising a wild-type TD from Arabidopsis thaliana. SEQ ID NOS:2 and 3 set forth nucleotide sequences, and amino acid sequences encoded thereby, comprising precursor proteins of differing lengths. SEQ ID NO:3 (see also FIG. 6b) encodes a 609 amino acid fusion or chimeric polypeptide of which 585 amino acid residues are encoded by mutant omr1 of Arabidopsis. That is, SEQ ID NO:3 encodes a mutant TD that is shorter than the full-length mutant TD shown in SEQ ID NO:2 by 7 amino terminal residues. Since transgenic plants transformed with pCM35s-omr1 were capable of expressing OMT resistance, then the 585 amino acid-long truncated precursor was fully capable of translocation from the cytoplasm to the chloroplast. SEQ ID NOS:4, 5 and 6 set forth sequences comprising three predicted mature proteins. SEQ ID NO:7 sets forth the putative regulatory site of an inventive mutated TD enzyme, and SEQ ID NOS:8 and 9 set forth regulatory regions harboring mutations in accordance with one aspect of the invention.

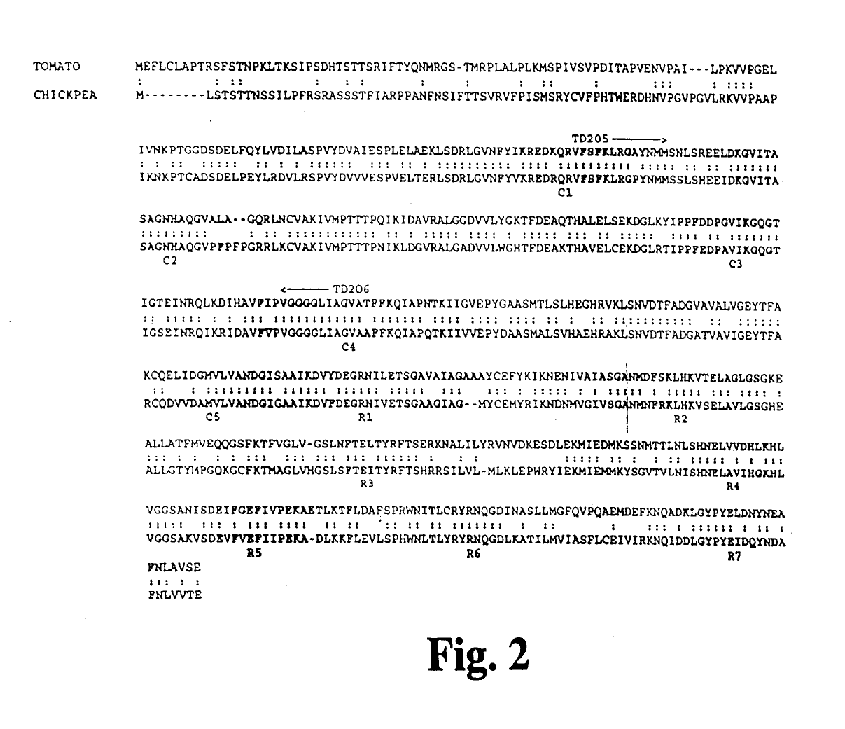

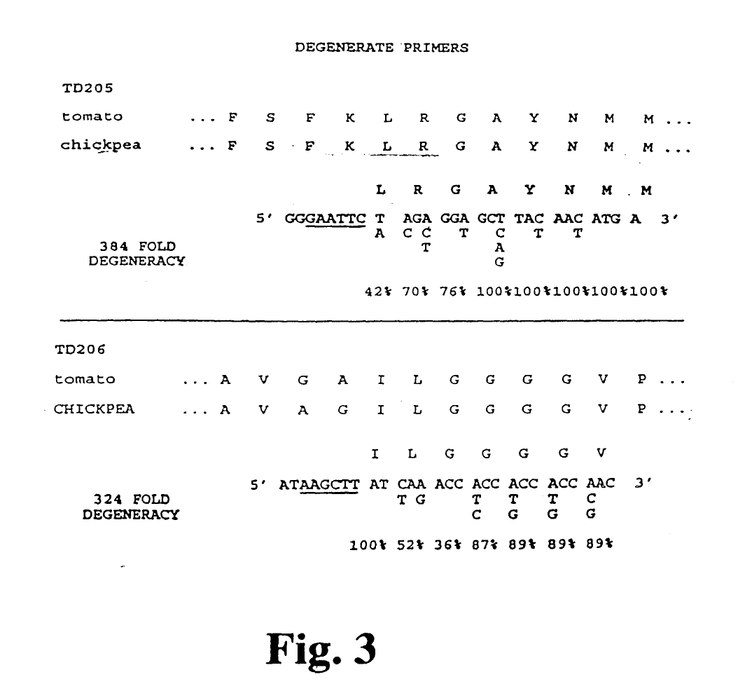

It is understood that the wild-type TD enzyme features dual functionality. Specifically, the TD enzyme has a catalytic site which is divided into catalytic regions C1-C5, as shown with respect to the analogous tomato TD enzyme and chickpea TD enzyme in FIG. 2. The catalytic site catalyzes the reaction of threonine to 2-oxobutyrate. TD also has a regulatory site which is divided into regulatory regions R1-R7, as shown in FIG. 2. The regulatory site is responsible for the feedback inhibition which occurs when the regulatory site binds to an inhibitor, in this case isoleucine.

The present application finds advantageous use in a wide variety of plants, as well as in a wide variety of microorganisms. With respect to plants, it is important to recognize that the TD enzyme functions in chloroplasts, and, therefore, that the polypeptide transcribed therefore is a precursor protein which includes a portion identified herein as a “chloroplast leader sequence.” For purposes of the present description, the term “chloroplast leader sequence” is used interchangeably with the term “transit peptide.” The chloroplast leader sequence is covalently bound to the “mature enzyme” or “passenger enzyme.” The term “precursor protein” is meant a polypeptide having a transit peptide and a passenger peptide covalently attached to each other. Typically, the carboxy terminus of the transit peptide is covalently attached to the amino terminus of the passenger peptide. The passenger peptide and transit peptide can be encoded by the same gene locus, that is, homologous to each other, in that they are encoded in a manner isolated from a single source. Alternatively, the transit peptide and passenger peptide can be heterologous to each other, i.e., the transit peptide and passenger peptide can be from different genes and/or different organisms. The terms “transit peptide,” “chloroplast leader sequence,” and “signal peptide” are used interchangeably to designate those amino acids that direct a passenger peptide to a chloroplast. By “mature peptide” or “passenger peptide” is meant a polypeptide which is found after processing and passing into an organelle and which is functional in the organelle for its intended purpose. Passenger peptides are originally made in a precursor form that includes a transit peptide and the passenger peptide. Upon entry into an organelle, the transit peptide portion is cleaved, thus leaving the “passenger” or “mature” peptide. Passenger peptides are the polypeptides typically obtained upon purification from a homogenate, the sequence of which can be determined as described herein.

The transit peptide may be derived from monocotyledonous or dicotyledonous plants upon choice of the artisan. DNA sequences encoding said transit peptides may be obtained from chloroplast proteins such as Δ-9 desaturase, palmitoyl-ACP thioesterase, β-KETOACYL-ACP synthase, oleyl-ACP thioesterase, chlorophyll a/b binding protein, NADPH+ dependent glyceraldehyde-3-phosphate dehydrogenase, early light inducible protein, clip protease regulatory protease, pyruvate orthophosphate dikinase, chlorophyll a/b binding protein, triose phosphate3-pohosphoglycerate phosphate translocator, 5-enol pyruval shikimate-e-phosphate synthase, dihydrofolate reductase, thymidylate synthase, acetyl-coenzyme A carboxylase, Cu/Zn superoxide dismutase, cystein synthase, rubisco activase, ferritin, granule bound starch synthase, pyrophosphate, glutamine synthase, aldolase, glutathione reductase, nitrite reductase, 2-oxoglutarate/malate translocator, ADP-glucose pyrophosphorylase, ferrodoxin, carbonic anhydrase, polyphenol oxidase, ferrodoxin NADP=oxidoreductase, platocyannin, glycerol-3-phosphate dehydrogenase, lipoxygenase, o-acetylserine (thiol)-lysase, acyl carrier protein, 3-deoxy-D-arabino-heptulosonate 7-phosphate synthase, chloroplast-localized heat shock protein, starch phosphorylase, pyruvate orthophosphate dikinase, starch glycosyltrtansferase, and the like, of which the transit peptide portion has been defined in GenBank.

In plants, the chloroplast leader sequence is used to direct the passenger protein to chloroplasts; however, they are typically cleaved and degraded upon entry of the passenger protein into the organelle of interest. Therefore, purification of a cleaved transit peptide from plant tissues is typically not possible. In some cases, however, transit peptide sequences can be determined by comparison of the precursor protein amino acid sequence obtained from the gene encoding the same to the amino acid sequence of the isolated passenger protein (mature protein). Furthermore, passenger protein sequences can also be determined from the transit peptide proteins associated therewith by comparison of sequences to other similar proteins isolated from different species. As exemplified herein, genes encoding precursor forms of mutated TD protein, disclosed as SEQ ID NO:2 and SEQ ID NO:3, when compared to wild type precursor and mature TD protein obtained from other species, can establish the expected sequence of the mature protein.

As previously discussed, the amino acid sequence and hence the nucleic acid sequence of a transit peptide can be determined in a variety of ways available to the skilled artisan. For example, passenger proteins of interest can be purified using a variety of techniques available to the person skilled in the art of protein biochemistry. Once purified, an amino terminal sequence of the protein can be determined using methods such as Edman degradation, mass spectroscopy, nuclear magnetic spectroscopy and the like. Using this information and the genetic code, standard molecular biology techniques can be employed to clone the gene encoding the protein as exemplified herein. Comparison of amino acid sequence determined from the cDNA to that obtained from the amino terminal sequence of the passenger protein can allow determination of the transit peptide sequence. In addition, many transit peptide sequences are available in the art and can easily be obtained form GenBank located in the Entrez Database at the National Center for Biotechnology Information web site.

The subject of transit peptides in plants has been extensively reviewed by Keegstra et al., (1989) (Cell, 56:247-253), which is incorporated herein by reference. Typically, there is very little primary amino acid sequence homology between different plant transit peptides. Even though passenger proteins may have amino acid and nucleic acid sequence similarities between cultivars, lines, and species, transit peptide may show very little sequence homology at any level. Furthermore, the length of transit peptides can vary, with some precursor proteins comprising transit peptide proteins with as few as about 10 amino acids while others can be about 150 amino acids or longer. Additional descriptions of transit peptide characteristics in plants and mechanisms associated therewith can be found in Ko and Ko, (1992) J. Biol. Chem. 267, 13910-13916; Bascomb et al. (1992) Plant Microb. Biotechnol. Res. Ser. 1:142-163; and Bakau et al., (1996) Trends in Cell Biol. 6:480-486; which are incorporated herein by reference.

In this regard, the first 90 amino acid residues in the N-terminal region of the Arabidopsis TD protein encoded by omr1 (in SEQ ID NO:2) represent an expected region comprising the transit peptide, as indicated by:

(i) the dissimilarity with the yeast, Salmonella and E. coli TD proteins,

(ii) the comparison of the sizes of TD of Arabidopsis, tomato, chickpea, yeast, Salmonella and E. coli, and

(iii) the amino acid composition which contains 12 proline residues and 33 other hydrophobic residues constituting a total of 50% hydrophobic residues.

Therefore, it is expected that the mature/passenger TD of Arabidopsis encoded by the omr1 locus, cleavage of the transit peptide may occur at the peptide bond between the alanine at residue 90 and the glutamic acid at residue 91, leaving behind a mature/passenger TD that starts at the glutamic acid at residue 91. As such, SEQ ID NO:4 identifies an expected mature TD for Arabidopsis that starts at the glutamic acid at residue 91 of SEQ ID NO:2 (clone 592). This expected mature TD polypeptide comprises 502 sequential amino acid residues.

The only two other higher plant TD genes that have been cloned to date are those of tomato (Samach A., Harven D., Gutfinger T., Ken-Dror S., Lifschitz E., 1991, Proc Natl Acad Sci USA 88:2678-2682) and chickpea (Jacob John S., Srivastava V., Guha-Mukherjee S., 1995, Plant Physiol 107:1023-1024). The lengths of the transit peptides of the tomato TD and chickpea TD were predicted to be the first −80 and 91 amino terminal residues, respectively, and the full length precursor proteins were reported to be 595 residues and 590 residues, respectively (Samach et al., 1991; Jacob John et al., 1995). In both tomato and chickpea, the amino-terminus of the TD protein contained a typical two-domain transit peptide consistent with chloroplast lumen targeting sequences (Keegstra K., Olsen L. J., Theg S. M., 1989, Chloroplast precursors and their transport across the membrane. Annu Rev Plant Physiol Plant Mol Biol 40:471-501). In tomato, the first domain at the amino-terminal (45 residues) of the transit peptide was rich in serine and threonine (33%) while the following sequence of 35 residues contained 8 regularly spaced proline and other hydrophobic residues (Samach et al., 1991). By sequencing the first ten amino-terminal residues of a purified tomato TD from flowers, Samach et al., (1991) found that lysine at residue 52 is the first amino acid at the amino-terminal end of the mature/passenger protein. According to Samach et al., (1991), the hydrophobic domain of the transit peptide of tomato TD is not cleaved and remains as part of the mature TD in the chloroplast. Samach et al., (1991) also explained that “it is possible that only a fraction of the tomato TD protein is cleaved at position 52, while the rest of the transit peptide is cleaved elsewhere and remain refractory to amino-terminal sequencing.” In chickpea, the first domain at the amino-terminal end of the transit peptide was deduced to be 45 residues and rich in threonine and serine (37%) while the remaining 46 residues contained 8 regularly spaced proline residues and 19 other hydrophobic residues (Jacob John et al., 1995). The cleavage site of the transit peptide of chickpea TD was not determined.

By analogy to tomato and chickpea, Arabidopsis TD also showed a typical two-domain transit peptide consistent with chloroplast lumen targeting sequences (as reviewed by Keegstra et al., 1989). The first 49 residues of the amino terminal end represented a domain that was rich in serine and threonine (31%) and other hydrophilic residues while the remaining 41 residues represented a second domain that contained 59% hydrophobic residues. The cleavage site of the transit peptide of Arabidopsis TD was not determined. Therefore, by analogy to tomato, it is expected that the cleavage site of the transit peptide of Arabidopsis TD may alternatively start at the lysine at residue 54 or at the lysine at residue 61. This is a presumptive cleavage site and one skilled in the art can readily determine the cleavage site in a similar fashion as in the case of tomato (Samach et al., 1991) by purifying Arabidopsis TD then sequencing the first ten amino acids in the amino-terminal end. Therefore, two additional sequences are provided as SEQ ID NOS:5 and 6 that alternatively identify two expected mature TD in Arabidopsis.

It is within the scope of the present invention to create chimeric polynucleotides encoding precursor proteins wherein a transit peptide of choice is in the proper reading frame with the mature coding sequence of mutated TD. As used herein, the terms “chimeric polynucleotide,” “chimeric DNA construct” and “chimeric DNA” are used to refer to recombinant DNA.

In creating a chimeric DNA construct encoding a transit peptide as disclosed herein, the transit peptide being heterologous to the mature, mutated TD, the DNA encoding the transit peptide is place 5′ and in the proper reading frame with the DNA encoding the mature, mutated TD protein. Placement of the chimeric DNA in correct relationship with promoter regulatory elements and other sequences as described herein can allow production of mRNA molecules that encode for heterologous precursor proteins. By “promoter regulatory element” is meant nucleotide sequence elements within a nucleotide sequence which control the expression of that nucleotide sequence. Promoter regulatory elements provide the nucleic acid sequences necessary for recognition of RNA polymerase and other transcriptional factors required for efficient transcription. Promoter regulatory elements are meant to include constitutive, tissue-specific, developmental-specific, inducible promoters and the like. Promoter regulatory elements may also include certain enhancer sequence elements that improve transcriptional efficiency. The mRNA can then be translated thus producing a functional heterologous precursor protein which can be delivered to the chloroplast. It is, of course, understood that a DNA construct may be made in accordance with the invention to include a promotor that is native to the gene of a selected species that encodes that species' TD precursor polypeptide. Uptake of the protein by the chloroplast and cleavage of the associated transit peptide can result in a chloroplast containing a mature, mutated form of TD, thus rendering the cell resistant to feedback inhibition which would normally inhibit cells containing only the wild-type TD protein.

The present invention, therefore, provides, in alternative aspects, a feedback insensitive TD comprising the amino acid sequence set forth in SEQ ID NO:2 or SEQ ID NO:3 (precursor polypeptides); set forth in SEQ ID NO:4, SEQ ID NO:5 or SEQ ID NO:6 (expected mature TD enzymes); SEQ ID NO:7 (an insensitive TD regulatory site); and set forth in SEQ ID NO:8 (regulatory region R4) or SEQ ID NO:9 (regulatory region R6). SEQ ID NO:7 or variants thereof as described above, may be operably coupled to a sequence encoding a TD catalytic site from a wide variety of species, including functionally similar variants thereof, to provide the advantageous result of the invention.

It is readily understood that, in the case of transforming prokaryotes, it is not necessary to include a transit peptide in the coding region of the vector. Rather, since such cells do not possess chloroplasts, an inventive DNA construct for transforming, for example, bacteria, may be made by simply attaching a start codon directly to, and in the proper reading frame with, a mature peptide. Of course, other elements are preferably present as described herein, such as a promoter upstream of the start codon and a termination sequence downstream of the coding region.

SEQ ID NOS:8 and 9 may also be operably coupled to a wide variety of sequences to provide insensitive TD enzymes, and therefore comprise certain preferred aspects of the invention. Substitutions giving rise to similar amino acid sequences, as described herein, are particularly applicable to SEQ ID NO:8, and the following sets forth a plurality of particularly preferred alternative sequences for SEQ ID NO:8 in accordance with the invention:

Val/Leu/Phe/Ile Asn/Asp/Glu/Ser Leu/Ile/Phe/Val/Gly Thr/Ser/Ala/Gly
Thr/His/Asp/ASn Ser/Asn/Asp/Ile Asp/Glu Leu/Met Val/Ala Lys/Val/Ala
Asp/Ile/Glu/Ser His Leu/Gly/Ile/Val Cys Tyr/His Leu/Met Met/Val
Gly Gly

The invention therefore also encompasses amino acid sequences similar to the amino acid sequences set forth herein that have at least about 50% identity thereto and that are insensitive to feedback inhibition by Ile. Preferably, inventive amino acid sequences have at least about 75% identity to these sequences, more preferably at least about 85% identity and most preferably at least about 95% identity.

Percent identity may be determined, for example, by comparing sequence information using the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981). Briefly, the GAP program defines identity as the number of aligned symbols (i.e., nucleotides or amino acids) which are the same, divided by the total number of symbols in the shorter of the two sequences. The preferred default parameters for the GAP program include: (1) a uniary comparison matrix (containing a value of 1 for identities and 0 for non-identities), and the weighted comparison matrix of Gribskov and Burgess, Nucl. Acids Res. 14:6745, 1986, as described by Schwartz and Dayhoff, eds., Atlas of protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

The invention also contemplates amino acid sequences having alternative mutations to those identified herein which also result in a feedback insensitive TD. For example, it is expected that the cys at position 499 and the his at position 544 in SEQ ID NO:2 could be substituted with alternative amino acids from the same amino acid group as cys and his, respectively (as described above) to provide an alternate inventive enzyme. Further, it is well within the purview of a person skilled in the art to engineer a feedback insensitive TD by providing a wild-type TD and substituting a highly conserved amino acid at a given location in the regulatory site with a diverse amino acid (i.e., one from a different amino acid group), and to assay the resulting enzyme for catalytic activity and feedback sensitivity. For example, a skilled artisan can alter the nucleotide sequence set forth in SEQ ID NO:1 by site-directed mutagenesis to provide a mutated sequence which encodes an enzyme having an alternate amino acid in a given location of the enzyme. Alternatively, a skilled artisan can synthesize an amino acid sequence having one or more additions, substitutions and/or deletions at a highly conserved location of the wild-type TD enzyme using techniques known in the art. Such variants, which exhibit functionality substantially similar to a polypeptide comprising the sequence set forth in SEQ ID NO:2, are included within the scope of the present invention.

Turning now to nucleotide sequences encoding inventive insensitive TD enzymes, nucleotide sequences encoding preferred feedback insensitive precursor TD of the species Arabidopsis thaliana are set forth in SEQ ID NOS:2 and 3 herein. The mutated polynucleotides set forth therein are referred to as omr1. omr1 has been found to be a dominant allele, this imparting significant value to the invention. It is of course not intended that the present invention be limited to this exemplary nucleotide sequence, but include sequences having substantial identity thereto and sequences which encode variant forms of insensitive TD as described above.

The term “nucleotide sequence,” as used herein, is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a functional polypeptide, such as, for example, an active enzyme. The process of encoding a specific amino acid sequence may involve DNA sequences having one or more base changes (i.e., insertions, deletions, substitutions) that do not cause a change in the encoded amino acid, or which involve base changes which may alter one or more amino acids, but do not eliminate the functional properties of the polypeptide encoded by the DNA sequence.

It is therefore understood that the invention encompasses more than the specific exemplary nucleotide sequence of omr1. For example, a nucleic-acid sequence encoding a variant amino acid sequence, as discussed above, is within the scope of the invention. Modifications to a sequence, such as deletions, insertions, or substitutions in the sequence which produce “silent” changes that do not substantially affect the functional properties of the resulting polypeptide molecule are expressly contemplated by the present invention. For example, it is understood that alterations in a nucleotide sequence which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product.

Nucleotide changes which result in alteration of the N-terminal and C-terminal portions of the polypeptide molecule would also not be expected to alter the activity of the polypeptide. In some cases, it may in fact be desirable to make mutations in the sequence in order to study the effect of alteration on the biological activity of the polypeptide. Each of the proposed modifications is well within the routine skill in the art.

In a preferred aspect, therefore, the present invention contemplates nucleotide sequences having substantial identity to the sequences set forth herein and variants thereof as described herein. The term “substantial identity” is used herein with respect to a nucleotide sequence to designate that the nucleotide sequence has a sequence sufficiently similar to a reference nucleotide sequence that it will hybridize therewith under moderately stringent conditions, this method of determining identity being well known in the art to which the invention pertains. Briefly, moderately stringent conditions are defined in Sambrook et al., Molecular Cloning: a Laboratory Manual, 2ed. Vol. 1, pp. 101-104, Cold Spring Harbor Laboratory Press (1989) as including the use of a prewashing solution of 5×SSC, 0.5% SDS, 1.0 mM EDTA (pH 8.0) and hybridization and washing conditions of about 55° C., 5×SSC. A further requirement of an inventive polynucleotide variant is that it must encode a polypeptide having similar functionality to the specific mutated TD enzymes recited herein, i.e., good catalytic functionality and insensitivity to feedback inhibition.

A suitable DNA sequence selected for use according to the invention may be obtained, for example, by cloning techniques using cDNA libraries corresponding to a wide variety of species, these techniques being well known in the relevant art. Suitable nucleotide sequences may be isolated from DNA libraries obtained from a wide variety of species by means of nucleic acid hybridization or PCR, using as hybridization probes or primers nucleotide sequences selected in accordance with the invention, such as those set forth in SEQ ID NOS:1-10; nucleotide sequences having substantial identity thereto; or portions thereof. Isolated wild-type sequences encoding TD may then be altered as provided by the present invention by site-directed mutagenesis.

Alternatively, a suitable sequence may be made by techniques which are also well known in the art. For example, nucleic acid sequences encoding enzymes of the invention may be constructed using standard recombinant DNA technology, for example, by cutting or splicing nucleic acids which encode cytokines and/or other peptides using restriction enzymes and DNA ligase. Alternatively, nucleic acid sequences may be constructed using chemical synthesis, such as solid-phase phosphoramidate technology. In preferred embodiments of the invention, polymerase chain reaction (PCR) is used to accomplish splicing of nucleic acid sequences by overlap extension as is known in the art.

Inventive DNA sequences can be incorporated into the genome of a plant or microorganism using conventional recombinant DNA technology, thereby making a transformed plant or microorganism having the excellent features described herein. In this regard, the term “genome” as used herein is intended to refer to DNA which is present in a plant or microorganism and which is heritable by progeny during propagation thereof. As such, an inventive transformed plant or microorganism may alternatively be produced by producing F1 or higher generation progeny of a directly transformed plant or microorganism, wherein the progeny comprise the foreign nucleotide sequence. Transformed plants or microorganisms and progeny thereof are all contemplated by the invention and are all intended to fall directly within the meaning of the terms “transformed plant” and “transformed microorganism.”

In this manner, the present invention contemplates the use of transformed plants which are selfed to produce an inbred plant. The inbred plant produces seed containing the gene of interest. These seeds can be grown to produce plants that express the protein of interest. The inbred lines can also be crossed with other inbred lines to produce hybrids. Parts obtained from the regenerated plant, such as flowers, seeds, leaves, branches, fruit, and the like are covered by the invention provided that said parts contain genes encoding and/or expressing the protein of interest. Progeny and variants, and mutants of the regenerated plants are also included within the scope of the invention.

In diploid plants, typically one parent may be transformed and the other parent is the wild type. After crossing the parents, the first generation hybrids (F1) are selfed to produce second generation hybrids (F2). Those plants exhibiting the highest levels of the expression can then be chosen for further breeding.

Genes encoding precursor mutated TD polypeptides, as disclosed herein as SEQ ID NO:2 and SEQ ID NO:3, can be used in conjunction with other plant regulatory elements to create plant cells expressing the polypeptides. By “expressing” as used herein, is meant the transcription and stable accumulation of mRNA inside a cell, the cell being of prokaryotic or eukaryotic origin. Furthermore, it is within the scope of the invention to place mutated mature TD from Arabidopsis into other species including monocotyledonous and dicotyledonous plants. In so doing, chimeric gene constructs encoding the mature, mutated TD proteins having transit peptides heterologous thereto (transit peptides from a different protein or species) can be used. Transit peptides of the present invention, when covalently attached to the mature, mutated TD protein, can provide intracellular transport to the chloroplast. In plants, a mutated mature form of TD found in a chloroplast of a cell renders the cell resistant to feedback inhibition and resistance to Ile structural analogs.

Generally, transformation of a plant or microorganism involves inserting a DNA sequence into an expression vector in proper orientation and correct reading frame. The vector may desirably contain the necessary elements for the transcription of the inserted polypeptide-encoding sequence. A wide variety of vector systems known in the art can be advantageously used in accordance with the invention, such as plasmids, bacteriophage viruses or other modified viruses. Suitable vectors include, but are not limited to the following viral vectors: lambda vector system gt11, gt10, Charon 4, and plasmid vectors such as pBI121, pBR322, pACYC177, pACYC184, pAR series, pKK223-3, pUC8, pUC9, pUC18, pUC19, pLG339, pRK290, pKC37, pKC101, pcDNAII, and other similar systems. The DNA sequences may be cloned into the vector using standard cloning procedures in the art, for example, as described by Maniatis et al., Molecular Cloning: A Laboratory Manual, Cold Springs Laboratory, Cold Springs Harbor, N.Y. (1982), which is hereby incorporated by reference in its entirety. The plasmid pBI121 is available from Clontech Laboratories, Palo Alto, Calif. It is understood that known techniques may be advantageously used according to the invention to transform microorganisms such as, for example, Agrobacterium sp., yeast, E. coli and Pseudomonas sp.

In order to obtain satisfactory expression of a nucleotide sequence which encodes an inventive feedback insensitive TD in a plant or microorganism, it is preferred that a promoter be present in the expression vector. The promoter is preferably a constitutive promoter, but may alternatively be a tissue-specific promoter or an inducible promoter. Preferably, the promoter is one isolated from a native gene which encodes a TD. Although promoters for certain classes of genes commonly differ between species, it is understood that the present invention includes promoters which regulate expression of a wide variety of genes in a wide variety of plant or microorganism species.

An expression vector according to the invention may be either naturally or artificially produced from parts derived from heterologous sources, which parts may be naturally occurring or chemically synthesized, and wherein the parts have been joined by ligation or other means known in the art. The introduced coding sequence is preferably under control of the promoter and thus will be generally downstream from the promoter. Stated alternatively, the promoter sequence will be generally upstream (i.e., at the 5′ end) of the coding sequence. The phrase “under control of” contemplates the presence of such other elements as may be necessary to achieve transcription of the introduced sequence. As such, in one representative example, enhanced production of a feedback insensitive TD may be achieved by inserting an inventive nucleotide sequence in a vector downstream from and operably linked to a promoter sequence capable of driving expression in a host cell. Two DNA sequences (such as a promoter region sequence and a feedback insensitive TD-encoding nucleotide sequence) are said to be operably linked if the nature of the linkage between the two DNA sequences does not (1) result in the introduction of a frame-shift mutation, (2) interfere with the ability of the promoter region sequence to direct the transcription of the desired nucleotide sequence, or (3) interfere with the ability of the desired nucleotide sequence to be transcribed by the promoter region sequence.

RNA polymerase normally binds to the promoter and initiates transcription of a DNA sequence or a group of linked DNA sequences and regulatory elements (operon). A transgene, such as a nucleotide sequence selected in accordance with the present invention, is expressed in a transformed cell to produce in the cell a polypeptide encoded thereby. Briefly, transcription of the DNA sequence is initiated by the binding of RNA polymerase to the DNA sequence's promoter region. During transcription, movement of the RNA polymerase along the DNA sequence forms messenger RNA (“mRNA”) and, as a result, the DNA sequence is transcribed into a corresponding mRNA. This mRNA then moves to the ribosomes of the cytoplasm or rough endoplasmic reticulum which, with transfer RNA (“tRNA”), translates the mRNA into the polypeptide encoded thereby.

It is well known that there may or may not be other regulatory elements (e.g., enhancer sequences) which cooperate with the promoter and a transcriptional start site to achieve transcription of the introduced (i.e., foreign) coding sequence. By “enhancer” is meant nucleotide sequence elements which can stimulate promoter activity in a cell such as those found in plants as exemplified by the leader sequence of maize streak virus (MSV), alcohol dehydrogenase intron 1, and the like. Also, the recombinant DNA will preferably include a transcriptional termination sequence downstream from the introduced sequence. It may also be desirous to use a reporter gene. In some instances, a reporter gene may be used with or without a selectable marker. Reporter genes are genes which are typically not present in the recipient organism or tissue and typically encode proteins resulting in some phenotypic change or enzymatic property. Examples of such genes are provided in K. Wising et al. (1988) Ann. Rev. Genetics, 22:421, which is incorporated herein by reference. Preferred reporter genes include the beta-glucuronidase (GUS) of the uidA locus of E. coli, the green fluorescent protein from the bioluminescent jellyfish Aequorea victoria, and the luciferase genes from firefly Photinus pyralis. An assay for detecting reporter gene expression may then be performed at a suitable time after the gene has been introduced into recipient cells. A preferred such assay entails the use of the gene encoding beta-glucuronidase (GUS) of the uidA locus of E. coli, as described by Jefferson et al., (1987 Biochem. Soc. Trans. 15, 17-19) to identify transformed cells.

Plant promoter regulatory elements from a wide variety of sources can be used efficiently in plant cells to express foreign genes. For example, promoter regulatory elements of bacterial origin, such as the octopine synthase promoter, the nopaline synthase promoter, the mannopine synthase promoter, and promoters of viral origin, such as the cauliflower mosaic virus (35S and 19S), 35T (which is a re-engineered 35S promoter, WO 97/13402 published Apr. 17, 1997) and the like may be used. Plant promoter regulatory elements include, but are not limited to, ribulose-1-5-bisphosphate (RUBP) carboxylase small subunit (ssu), beta-conglycinin promoter, beta-phaseolin promoter, ADH promoter, heat-shock promoters, and tissue-specific promoters.

Other elements such as matrix attachment regions, scaffold attachment regions, introns, enhancers, polyadenylation sequences, and the like, may be present and thus may improve the transcription efficiency or DNA integration. Such elements may or may not be necessary for DNA function, although they can provide better expression or functioning of the DNA by affecting transcription, mRNA stability, and the like. Such elements may be included in the DNA as desired to obtain optimal performance of the transformed DNA in the plant. Typical elements include, but are not limited to, Adh-intron 1, Adh-intron 0.6, the alfalfa mosaic virus coat protein leader sequence, the maize streak virus coat protein leader sequence, as well as others available to a skilled artisan.

Constitutive promoter regulatory elements may be used thereby directing continuous gene expression in all cell types at all times (e.g., actin, ubiquitin, CaMV 35S, and the like). Tissue specific promoter regulatory elements are responsible for gene expression in specific cell or tissue types, such as the leaves or seeds (e.g., zein, oleosin, napin, ACP, globulin, and the like) and these may alternatively be used.

Promoter regulatory elements may also be active during a certain stage of the plants' development as well as active in plant tissues and organs. Examples of such include, but are not limited to, pollen-specific, embryo-specific, corn silk-specific, cotton fiber-specific, root-specific, seed endosperm-specific promoter regulatory elements, and the like. Under certain circumstances, it may be desirable to use an inducible promoter regulatory element, which is responsible for expression of genes in response to a specific signal, such as, for example, physical stimulus (heat shock genes), light (RUBP carboxylase), hormone (Em), metabolites, chemicals and stress Other desirable transcription and translation elements that function in plants may also be used. Numerous plant-specific gene transfer vectors are known in the art.

Once the DNA construct of the present invention has been cloned into an expression vector, it may then be transformed into a host cell. In addition to numerous technologies for transforming plants, the type of tissue which is contacted with foreign polynucleotides may vary as well. Plant tissue suitable for transformation of a plant in accordance with certain preferred aspects of the invention include, for example, whole plants, leaf tissues, flower buds, root tissues, callus tissue types I, II and III, embryogenic tissue, meristems, protoplasts, hypocotyls and cotyledons. It is understood, however, that this list is not intended to be limiting, but only to provide examples of plant tissues which may be advantageously transformed in accordance with the present invention. A wide variety of plant tissues may be transformed during dedifferentiation using appropriate techniques described herein.

Transformation of a plant or microorganism may be achieved using one of a wide variety of techniques known in the art. The manner in which the transcriptional unit is introduced into the plant host is not critical to the invention. Any method which provides efficient transformation may be employed. One technique of transforming plants with a DNA construct inaccordance with the present invention is by contacting the tissue of such plants with an inoculum of bacteria transformed with a vector comprising the DNA construct. Generally, this procedure involves inoculating the plant tissue with a suspension of bacteria and incubating the tissue for about 48 to about 72 hours on regeneration medium without antibiotics at about 25-28° C. Bacteria from the genus Agrobacterium may be advantageously utilized to transform plant cells. Suitable species of such bacterium include Agrobacterium tumefaciens and Agrobacterium rhizogenes. Agrobacterium tumefaciens (e.g., strains LBA4404 or EHA105) is particularly useful due to its well-known ability to transform plants. Another technique which may advantageously be used is vacuum-infiltration of flower buds using Agrobacterium-based vectors.

Various methods for plant transformation include the use of Ti or Ri-plasmids and the like to perform Agrobacterium mediated transformation. In many instances, it will be desirable to have the construct used for transformation bordered on one or both sides by T-DNA borders, more specifically the right border. This is particularly useful when the construct uses Agrobacterium tumefaciens or Agrobacterium rhizogenes as a mode for transformation, although T-DNA borders may find used with other modes of transformation. Where Agrobacterium is used for plant transformation, a vector may be used which may be introduced into the host for homologous recombination with T-DNA or the Ti or Ri plasmid present in the host. Introduction of the vector may be performed via electroporation, tri-parental mating and other techniques for transforming gram-negative bacteria which are known to those skilled in the art. The manner of vector transformation into the Agrobacterium host is not critical to the invention.

In some cases where Agrobacterium is used for transformation, the expression construct being within the T-DNA borders will be inserted into a broad spectrum vector such as pRK2 or derivatives thereof as described in Ditta et al. (PNAS USA (1980) 77:7347-7351 and EPO 0 120 515), which are incorporated herein by reference. Explants may be combined and incubated with the transformed Agrobacterium for sufficient time to allow transformation thereof. After transformation, the Agrobacteria and plant cells are cultured with the appropriate selective medium. Once calli are formed, shoot formation can be encouraged by employing the appropriate plant hormones according to methods well known in the art of plant tissue culturing and plant regeneration. However, a callus intermediate stage is not always-necessary. After shoot formation, said plant cells can be transferred to medium which encourages root formation thereby completing plant regeneration. The plants may then be grown to seed and the seed can be used to establish future generations. Regardless of transformation technique, the polynucleotide of interest is preferably incorporated into a transfer vector adapted to express the polynucleotide in a plant cell by including in the vector a plant promoter regulatory element, as well as 3′ non-translated transcriptional termination regions such as Nos and the like.

Plant RNA viral based systems can also be used to express genes for the purposes disclosed herein. In so doing, the chimeric genes of interest can be inserted into the coat promoter regions of a suitable plant virus under the control of a subgenomic promoter which will infect the host plant of interest. Plant RNA viral based systems are described, for example, in U.S. Pat. Nos. 5,500,360; 5,316,931 and 5,589,367, each of which is hereby incorporated herein by reference in its entirety.

Another approach to transforming plant cells with a DNA sequence selected in accordance with the present invention involves propelling inert or biologically active particles at plant tissues or cells. This technique is disclosed in U.S. Pat. Nos. 4,945,050, 5,036,006 and 5,100,792, all to Sanford et al., which are hereby incorporated by reference. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector. Alternatively, the target cell can be surrounded by the vector so that the vector is carried into the cell by the wake of the particle. Biologically active particles (e.g., dried yeast cells, dried bacterium or a bacteriophage, each containing DNA material sought to be introduced) can also be propelled into plant cells. It is not intended, however, that the present invention be limited by the choice of vector or host cell. It should of course be understood that not all vectors and expression control sequences will function equally well to express the DNA sequences of this invention. Neither will all hosts function equally well with the same vector expression system. However, one of skill in the art may make a selection among vectors, expression control sequences, and hosts without undue experimentation and without departing from the scope of this invention.

An isolated DNA construct selected in accordance with the present invention may be utilized in an expression vector to transform a wide variety of plants, including monocots and dicots. The invention finds advantageous use, for example, in transforming the following plants: rice, wheat, barley, rye, corn, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane. Additional literature describing plant and/or microorganism transformation includes the following, each of which is incorporated herein by reference in its entirety: Zhijian Li et al. “A Sulfonylurea Herbicide Resistance Gene from Arabidopsis thaliana as a New Selectable Marker for Production of Fertile Transgenic Rice Plants” Plant Physiol. 100, 662-668 (1992); Parsons et al. (1997) Proc. Natl. Acad. Sci. USA 84:4161-4165; Daboussi et al. (1989) Curr. Genet. 15:453-456; Leung et al. (1990) Curr. Genet. 17:409-411; Koetter et al., “Isolation and characterization of the Pichia stipitis xylitol gehydrogenase gene, XYL2, and construction of a xylose-utilizing Saccharomyces cerevisiae transformant,” Curr. Genet., 18:493-500 (1990); Strasser et al., “Cloning of yeast xylose reductase and xylitol dehydrogenase genes and their use,” German patent application (1990); Hallborn et al., “Xylitol production by recombinant Saccharomyces cerevisiae,” Bio./Technol., 9:1090 (1991); Becker and Guarente, “High efficiency transformation of yeast by electroporation,” Methods in Enzymol. 194:182-186 (1991); Ammerer, “Expression of genes in yeast using the ADC1 promoter,” Methods in Enzymol. 101:192-201 (1983); Sarthy et al., “Expression of the E. coli xylose isomerase gene in S. cerevisiae,” Appl. Environ. Microb., 53: 1996′-2000 (1987); U.S. Pat. Nos. 4,945,050, 5,141,131, 5,177,010, 5,104,310, 5,149,645, 5,469,976, 5,464,763, 4,940,838, 4,693,976, 5,591,616, 5,231,019, 5,463,174, 4,762,785, 5,004,863, 5,159,135, 5,302,523, 5,464,765, 5,472,869, 5,384,253; European Patent Application Nos. 0131624B 1, 120516, 159418B1, 176112, 116718, 290799, 320500, 604662, 627752, 0267159, 0292435; WO 87/06614; WO 92/09696; and WO 93/21 335.

Those skilled in the art will recognize the commercial and agricultural advantages inherent in plants transformed to express feedback insensitive TD. Such plants have the improved ability to synthesize lie and, therefore, are expected to be more valuable nutritionally, compared to a corresponding non-transformed plant. Further, certain intermediates of the Ile biosynthetic pathway have significant commercial value, and production of these intermediates is advantageously increased in a transformant in accordance with the invention. For example, 2-oxobutyrate, the reaction product of the reaction catalyzed by TD, is known to be a precursor for the production of polyhydroxybutyrate in plants that have been genetically engineered using techniques known in the art to include bacterial genes necessary to produce polyhydroxybutyrate. Polyhydroxybutyrate is a desired biopolymer in the plastic industry because it may be biologically degraded. Because plants and microorganisms transformed in accordance with the invention feature increased production of 2-oxobutyrate, such plants and/or microorganisms may be advantageously utilized by plastic manufacturers in this manner. For example, plants that overproduce 2-oxobutyrate would be ideal for metabolic engineering by bacterial genes for polyhydroxybutyrate production because the overproduction of 2-oxobutyrate would provide plenty of substrate for both the natural Ile biosynthetic pathway and the engineered polyhydroxybutyrate pathway.

Perhaps the most significant advantage of the present invention is that an inventive nucleotide sequence may be used in an expression vector as a selectable marker. In this aspect of the invention, an inventive nucleotide sequence is incorporated into a vector such that it is expressed in a cell transformed thereby, along with a second pre-selected nucleotide sequence (i.e., the primary sequence) which is desired to be incorporated into the genome of the target cell. In this inventive selection protocol, successful transformants will not only express the primary sequence, but will also express a feedback insensitive TD. Thus, once the recombinant DNA is introduced into the plant tissue or microorganism, successful transformants can be screened in accordance with the invention by growing the plant or microorganism in a substrate comprising a toxic Ile analog, such as, for example, OMT (termed “toxic substrate” herein). The Ile structural analog is toxic to wild-type TD, and only the successful transformants, i.e., those expressing feedback insensitive TD, will live, grow and/or proliferate in the toxic substrate.

In this manner, omr1 is also an excellent biochemical marker to be used in experiments of genetic engineering of bacteria replacing the traditionally used and environmentally-hazardous antibiotic-resistant genes (such as ampicillin- and kanamycin-resistant marker genes), omr1 is very environmentally friendly and poses no risk to human health when included in a transformant, because it does not have an ortholog in humans. Humans do not synthesize isoleucine and may only obtain it by digesting food.

Based upon the advantageous features of the invention, there is also provided a novel herbicide system. In accordance with this system, agriculturally valuable plant lines comprising an expressible nucleotide sequence encoding an insensitive TD (“transformed plant line”) are grown in a substrate and an Ile structural analog selected in accordance with the invention is contacted with the substrate or with the plants themselves. As a result, only the transformed plants will continue to grow and other plants contacted with the analog will die.

The invention will be further described with reference to the following specific Examples. It will be understood that these Examples are illustrative and not restrictive in nature. Restriction enzyme digestions, phosphorylations, ligations and bacterial transformations were done as described in Sambrook et al., Molecular Cloning: A Laboratory Manual, Second Edition (1989) Cold Spring Harbor Laboratory Press. Plant transformations were done according to Bent et al. “RPS2 of Arabidopsis thaliana: A leucine-rich repeat class of plant disease resistance genes.” Science 265:1856-1860 (1994). Each reference is incorporated herein by reference in its entirety.

### EXAMPLE ONE

As reported in Mourad G, King J (1995) L-O-methylthreonin-resistant mutant of Arabidopsis defective in isoleucine feedback regulation. Plant Physiol 107:43-52, the mutated line GM11b of Arabidopsis thaliana was obtained, using EMS-mutagenesis, by selection in the presence of the toxic Ile structural analog, L-O-methylthreonine (OMT). The basis of mutant selection was that OMT is incorporated into cellular proteins in place of Ile, causing loss of protein function and, thus, cell death. GM11b was rescued because of a dominant mutation in the single gene omr1 which encodes TD. The mutation in the omr1 gene causes TD from GM11b to be insensitive to feedback control by Ile. TD activity in extracts from GM11b plants was about 50-fold more resistant to feedback inhibition by Ile than TD in extracts from wild type plants. The loss of Ile feedback sensitivity in GM11b led to a 20-fold overproduction of free Ile when compared to the wild type. This overproduction of Ile in GM11b had no effect on plant growth or reproduction.

### EXAMPLE TWO

### Cloning, Sequencing and Testing omr1 as a Selectable Marker in Genetic Engineering Experiments

1. The construction of a cDNA Library from GM11b (omr1/omr1):

Total RNA was extracted from 16-day-old GM11b (omr1/omr1) plants that were germinated in a minimal agar medium supplemented with 0.2 mM MTR. Poly(A) RNA (mRNA) was extracted from the total RNA and complementary DNA (cDNA) was synthesized using reverse transcriptase. The cDNA library was synthesized using the ZAP-cDNA synthesis kit of Stratagene. To prime the cDNA synthesis, a 50-base oligonucleotide linker primer containing an Xho I site and an 18-base poly(dT) was used. A 13-mer oligonucleotide adaptor containing an Eco RI cohesive end was ligated to the double stranded cDNA molecules at the 5′ end. This allowed unidirectional cloning of the cDNA molecules, in the sense orientation, into the Eco RI and Xho I sites of the Uni-ZAP XR vector of Stratagene. The recombinant X phage library was amplified using the XL1-Blue MRF′ E. coli host cells yielding a titer 6.8×10⁹ pfu/ml. The average size insert was approximately 1.4 kb. This was calculated from PCR analysis of 20 random, clear plaques isolated from the amplified library. The Uni-ZAP XR vector contains the pBluescript SK(−) plasmid containing the N-terminus of the lacZ gene. To excise the pBluescript phagemid, containing the cloned cDNA insert, the ExAssist/SOLR system provided by Stratagene was used. This allowed the rescue of the cDNA inserts from the positive X clones in pBluescritpt SK plasmids in a single step.

2. The isolation of a Small TD-DNA Fragment to Use as a Homologous Probe:

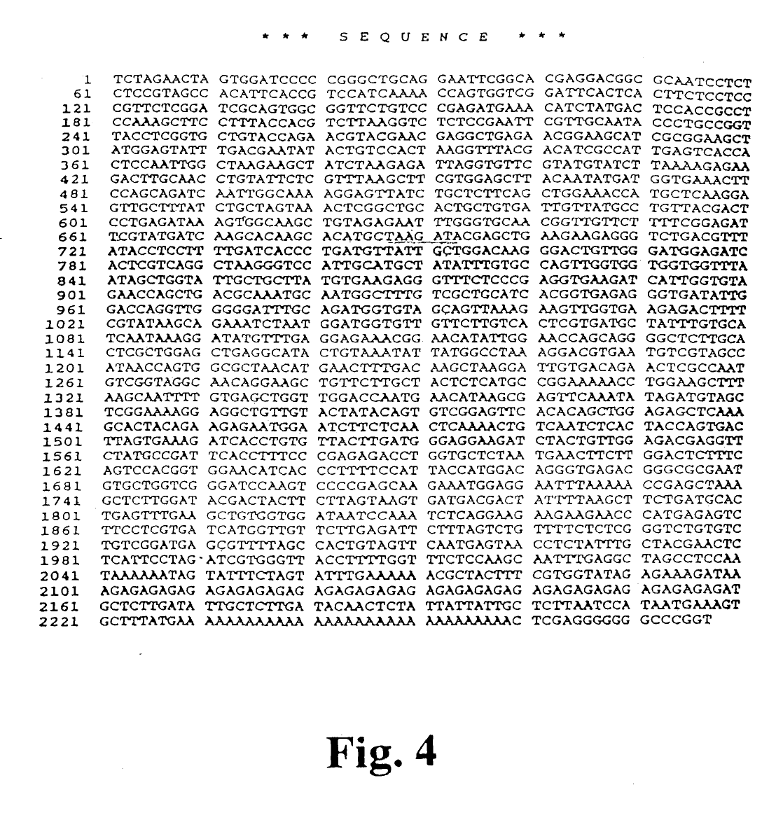

To isolate the omr1 gene encoding TD from the cDNA library of the line GM11b, a homologous oligonucleotide, isolated from Arabidopsis DNA, was used as a probe against the cDNA library. Taking into consideration that TD is conserved in a variety of organisms, degenerate primers were designed from conserved amino acid regions of TD. Such conserved regions were identified by aligning the amino acid sequence of TD from chickpea and tomato. FIG. 2 shows the location of the conserved amino sequences in tomato and chickpea and also the location of the degenerate oligonucleotide primers TD205 and TD206 that were designed to isolate a TD-DNA fragment from Arabidopsis. FIG. 4 shows the structure and degree of degeneracy of the PCR oligonucleotide primers, TD205 (the 5′ end primer) and TD206 (the 3′ end primer). Both primers TD 205 and TD 206 were designed to accommodate the Arabidopsis codon usage bias. Primer TD 205 had 384-fold degeneracy and was a 28-mer anchored with an Eco RI site starting 2 bases downstream from the first nucleotide at the 5′ end of the primer. TD 206 had 324-fold degeneracy and was a 28-mer anchored with a Hind III site starting 2 bases downstream from the first nucleotide at the 5′ end of the primer.

Genomic DNA was isolated from GM11b and used as a template in a PCR amplification with the primers TD205 and TD 206. A 438 bp fragment was amplified. The fragment was cloned into the Eco RI-Hind III sites of the plasmid pGEM3Zf(+). The fragment was sequenced to completion using the dideoxy chain termination method and the sequenase kit of USB. The fragment showed a putative 280 bp intron. The remaining 158 bp of the PCR-fragment had 60.1% identical nucleotide sequence with the chickpea TD gene. To eliminate the putative intron sequences, a second pair of primers TD 211 and TD212 were designed and used in a PCR reaction with the 438 bp fragment as a template. A DNA fragment of about 100 bp length, containing exon sequences, was amplified and purified. This was the homologous probe used for screening the cDNA library constructed from GM11b.

3. Screening the cDNA Library of GM11b:

The 100 bp PCR-fragment was labeled with [α-³²P]dCTP (3000 Ci/mmol) using random priming (prime-a gene labeling kit of Promega) and used as a probe to screen plaque lifts (two replicas per plate) of the plated GM11b cDNA library. Hybridization was done at 42° C. in formamide for 2 days. The nylon membranes containing the plaque lifts were washed 3×at room temperature (25° C.) in 7×SSPE and 0.5%SDS for 5 minutes. The nylon membranes were then put on X-ray film and exposed for 1 day. Two plaques hybridized and showed signal on the X-ray films of the two replicas taken from the same plate. At the site of positive hybridization, plugs were cut out of the agar plate and put in 1 ml of SM buffer with 20 μL chloroform. A secondary, tertiary and quaternary screening was performed until about 90% of the plaques on the plate showed a strong signal on the X-ray film of both replicas of the same plate. A well isolated plaque representing each clone was cut out from the plate and put in SM buffer. The phage eluate was infected with the ExAssist helper phage to excise the pBluescript SK plasmid containing the cDNA insert and the resulting recombinant bacteria was plated on media with ampicillin (60 μg/ml). A few bacterial colonies were selected, plasmid DNA was prepared then digested with Eco RI and Xho I to release the inserts. A Southern blot was prepared from the plasmid digests and probed with the ³²P-labelled 100 bp TD fragment. All the clones, descendants from the two phage clones, showed very strong signal. This was a strong indication that the isolated clones contained the TD from the line GM11b. One clone was named TD23 and was selected for DNA sequencing. The size of the cDNA insert in clone TD23 was 2229 nucleotides.

4. Sequencing of the 2229 bp Fragment of the Clone TD23:

Sequencing of the cDNA insert of clone TD23 was performed by the dideoxy chain termination method using the sequenase kit of USB. To start the sequencing project, an oligonucleotide primer complementary to the T3 promoter of pBluescript SK was synthesized and used to obtain the sequence of the first few nucleotides of the insert. This sequence, 30 nucleotides, included the multiple cloning site downstream of the T3 promoter. The start of the cDNA sequence was immediately following the Eco RI site which starts at position 31. DNA sequencing was also performed on the opposite strand starting from the 3′ end and using the T7 promoter of the pBluescript SK. Both strands of the TD 23 insert were sequenced to completion using a set of oligonucleotide primers designed from the DNA revealed after each sequencing reaction. A total of 19 oligonucleotide primers were synthesized and used in sequencing the cDNA insert.

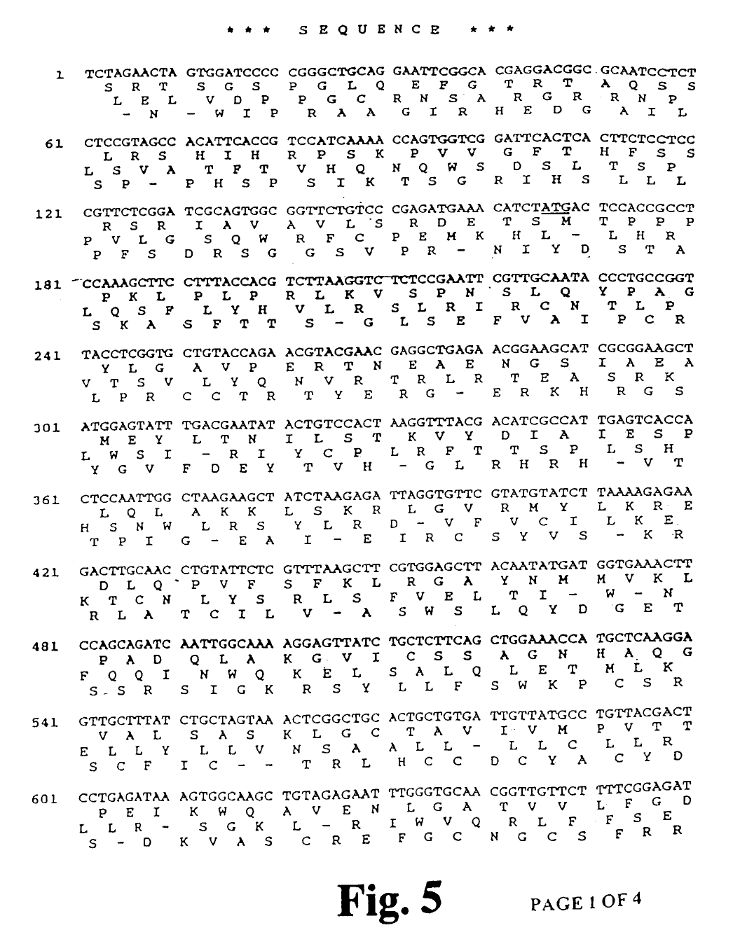

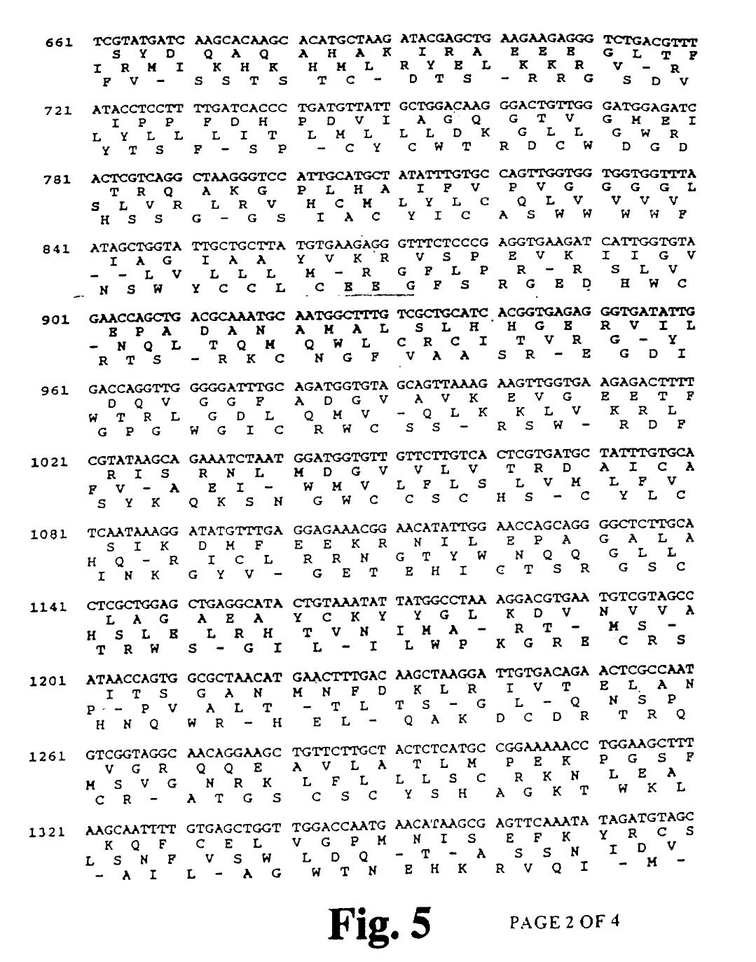

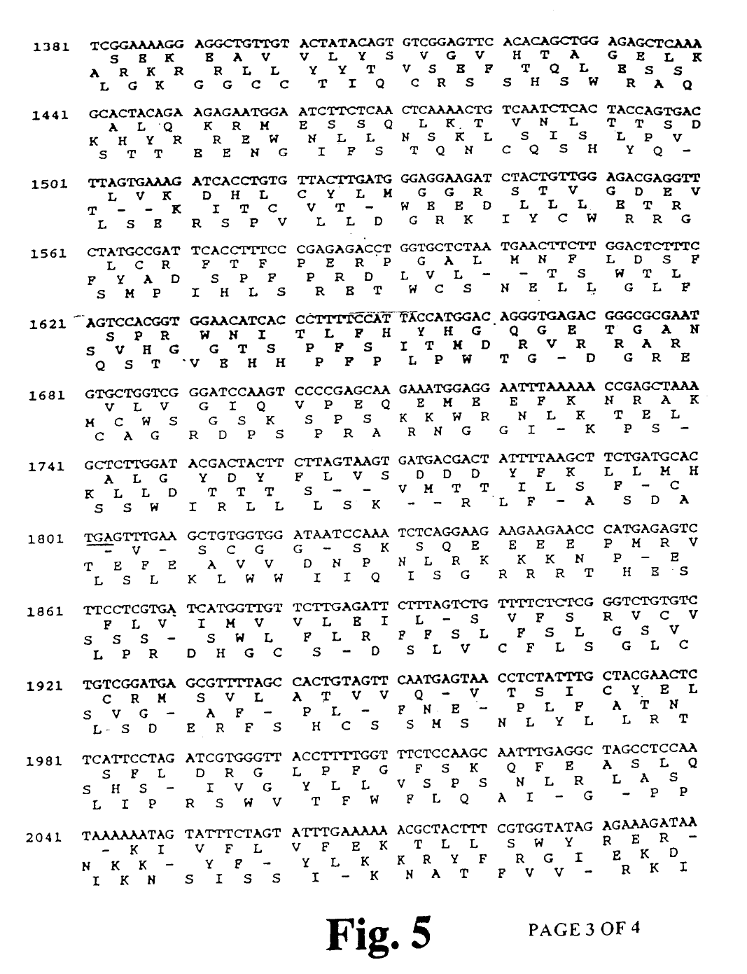

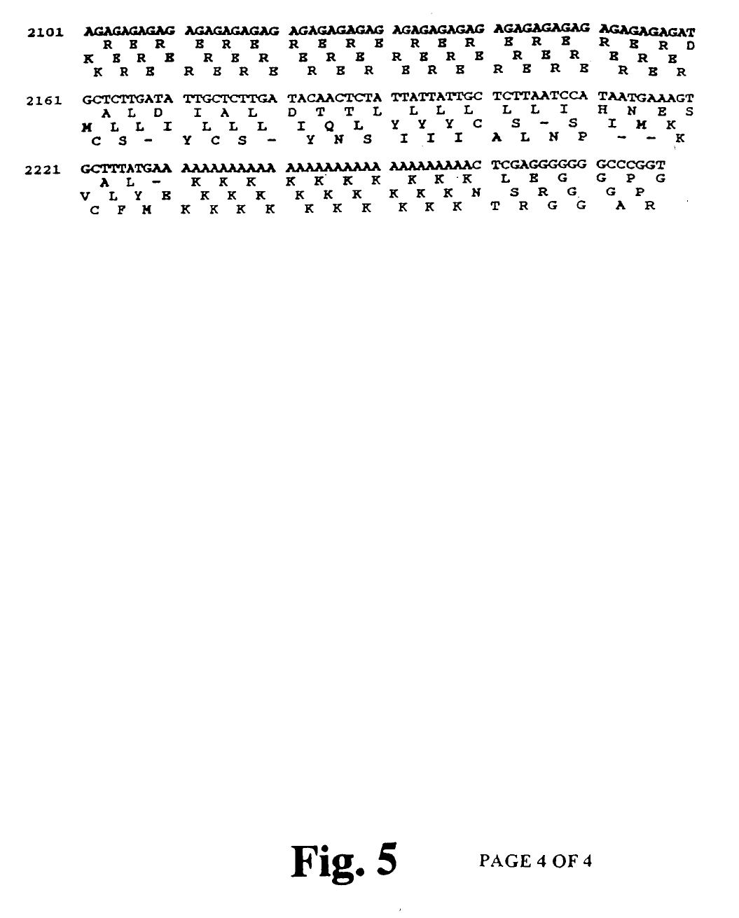

The total length of the sequenced fragment was 2277 nucleotides of which 2229 were the cDNA insert. Of the remaining 48 nucleotides, 2277-2229, 31 nucleotides were the multiple cloning site between the T3 promoter and the Eco RI site at the 5′ end of the insert and 17 nucleotides were multiple cloning site between the T7 promoter and Xho I site at the 3′ end of the insert (FIG. 4). FIG. 5 shows the nucleotide sequence and the predicted amino acid sequence of clone 23 as isolated from the cDNA library constructed from line GM11b of Arabidopsis (omr1/omr1). The TD insert in clone 23 is in pBluescript vector between the Eco RI and Xho I sites. An open reading frame (top reading frame) was observed which showed an ATG codon at nucleotide 166 and a termination codon at nucleotide 1801. The total cDNA insert in clone 23 is 1758 nucleotides (including the stop codon) encoding a polypeptide of 585 amino acids. FIG. 4 shows the DNA sequence of clone 23 and FIG. 5 shows the DNA sequence and the open reading frame with the predicted amino acid sequence encoded by the cDNA insert. The predicted amino acid sequence encoded by the TD 23 cDNA gene shared greater than 50% identity with the amino acid sequence of TD of potato and tomato respectively. This was strong evidence that the cDNA insert of the clone TD23 is indeed the gene encoding threonine dehydratase/deaminase, omr1, of the L-O-methylthreonine-resistant line GM1 lb of Arabidopsis thaliana.

5. Test of Functionality of the cDNA Insert (omr1) Encoding TD of Arabidopsis:

To test that the cloned cDNA insert of the clone TD 23 is indeed encoding a functional threonine dehydratase/deaminase, a complementation test was performed. The E. coli strain TGXA is an auxotroph with a deletion in the ilvA gene encoding threonine dehydratase/dearminase. Fisher K E, Eisenstein e (1993) An efficient approach to identify ilva mutations reveals an amino-terminal catalytic domain in biosynthetic threonine deaminase from Escherichia coli. J Bacteriol 175:6605-6613. This strain cannot grow on a minimal medium without supplementation with Ile. This strain was a generous gift from Drs. Kathryn E. Fisher and Edward Eisenstein, University of Maryland Baltimore County, Maryland.

First complementation experiments were done to test the ability of omr1 to revert the bacterial Ile auxotroph TGXA to prototrophy. This was done by transforming TGXA with pGM-td23, containing the cDNA insert omr1 in pBluescript SK under the control of the T3 promoter. In addition, the cDNA insert containing omr1 was subcloned in two different prokaryotic expression vectors. An Xba I-Xho I fragment, containing the cDNA sequence of omr1, was excised from pGM-td23 and cloned into Xba 1-Sal I linearized prokaryotic expression vectors pTrc99A and pUCK2. In pTrc99A, omr1 was cloned in front of the lacZ IPTG-inducible promoter while in pUCK2, omr1 was cloned in front of a constitutive promoter. Xho I and Sal I cohesive termini are compatible and therefore allowed the ligation of the inserts into the expression vectors. The recombinant vectors pTrc-td23, pUCK-td23 or pBluescript-td23 all containing full length omr1 were transformed into the strain TGXA and plated on minimal media without supplementation. All of the three constructs were able to revert Ile auxotrophy of the host TGXA to prototrophy. These experiments confirmed that omr1 encoding Arabidopsis thaliana (line GM11b) TD is functional and able to unblock the Ile biosynthetic pathway of the E. coli strain TGXA.

In the second complementation experiment, the E. coli prototroph host DH5α was transformed with pTrc-td23 or pUCK-td23 and plated on minimal medium supplemented with varying concentrations of the toxic analog L-O-methylthreonine. Both of the constructs were able to confer upon DH5α resistance to 30 μM L-O-methylthreonine. No bacterial colonies grew on plates containing untransformed DH5a. This result provided strong evidence that the mutated omr1 gene of the line GM11b of Arabidopsis is able to confer resistance to L-O-methylthreonine present in the growth medium. Therefore omr1 provides a new environmentally friendly selectable marker for genetic transformation of bacteria.

6. Construction of the pCM35S-omr1 Expression Vector for Plant Transformation:

The strategy for cloning the omr1. allele into a plant expression vector was as follows:

A. The coding region of omr1 allele was excised from pGM-td23 as an Xba I-Kpn I fragment.

B. The 500 bp CaMV 35S promoter was cleaved out of the vector pBI121.1 (Jefferson et al., 1987) with Hind III and Bam HI. The pBIN19 vector was linearized by Hind III and Bam HI then ligated to the CaMV 35S promoter so as to place the promoter into the multiple cloning site in the correct orientation. This vector was named pCM35S.

C. The plasmid pCM35S was digested with Xba I-Kpn I and the omr1 fragment isolated in step A was cloned into the Xba I-Kpn I sites placing the omr1 coding sequence in front of the CaMV 35S promoter and creating a plasmid with the kanamycin resistance gene (NOS:NPT11:NOS) close to the right border RB of the T-DNA region of the Ti plasmid and 35S:omr1 downstream and close to the left border LB of the T-DNA region of the Ti plasmid. This plasmid was named pCM35S-omr1-nos (ca. 13 kb).

D. The NOS terminator of pBIN19 was PCR-amplified using a pair of oligonucleotide primers, the 5′ primer was anchored with an Xba I site and the 3′ primer was anchored with a Sal I site. PCR amplification yielded a 300 bp NOS terminator fragment.

E. To clone a NOS terminator to the 3′ end of the omr1 gene, the recombinant plasmid pCM35S-omr1-nos was digested with Nhe Iand Xho 1. This yielded three fragments:

(i) a 5 kb Nhe I-Nhe I fragment containing part of the NOS promoter of the NPTII gene, the 35S promoter and the full length omr1 cDNA except 200 bp of non-translated sequences at the 3′ end which include the poly A tail.

(ii) a 200 bp Nhe I-Xho I fragment containing the 200 bp fragment mentioned in (i) and that contained the poly A tail and non-translated sequences at the 3′ end of omr1.

(iii) an 8 kb Nhe I-Xho I fragment containing the 5′ end NOS promoter of the NPTII gene and the remaining sequences outside LB and RB of the pCM35S-omr1-nos.

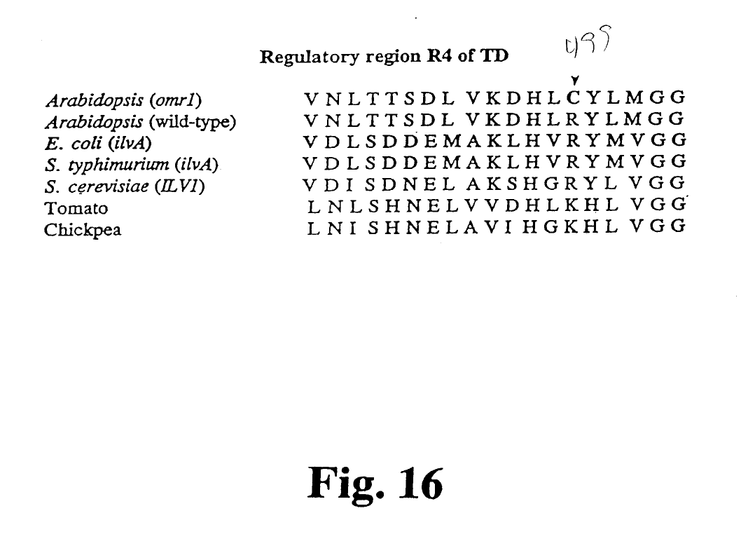

F. To clone the NOS terminator immediately downstream from the omr1 gene in pCM35S-omr1-nos, a triple ligation was performed including the 5 kb Nhe I-Nhe I fragment containing part of the NOS promoter of the NPTII gene mentioned above in E(i), the 300 bp Xba I-Sal I NOS terminator fragment mentioned in C, and the 8 kb Nhe I-Xho I fragment containing the 5′ end NOS promoter of the NPTII gene and the remaining sequences outside LB and RB of the pCM35S-omr1-nos. The result of this triple cloning was the ligation of the 5 kb fragment at one Nhe I end (the NOS promoter end) to the Nhe I site of the 8 kb fragment (Nhe I/Nhe I) and the other Nhe I end (at the 3′ end of the omr1 coding sequence) of the 5 kb fragment was ligated to the Xba I (isoschizomer) of the 300 bp NOS terminator fragment. The Sal I end of the 300 bp NOS terminator was ligated to the Xho I (isoschizomer) end of the 8 kb fragment. This generated the recombinant plasmid pCM35S-omr1 containing the omr1 gene driven by the CaMV 35S promoter and terminated by the NOS terminator and the kanamycin resistance gene (NOS promoter:NPTII:NOS:terminator) between the LB and RB (FIG. 16). To confirm the cloning of the three fragments in the proper orientation, a diagnostic digestion with Xba I & Kpn I produced a 2.3-2.4 kb fragment. The plasmid pCM35S-omr1 therefore contained two constructs that could be expressed in plants, the CaMV35S:omr1:NOS terminator expressing L-O-methylthreonine-resistance and the NOS promoter:NPT11:NOS terminator expressing kanamycin-resistance.

7. Plant Transformation Using pCM35S-omr1:

Using the vacuum infiltration method of Bent et al. (1994), L-O-methylthreonine-sensitive Arabidopsis thaliana Columbia wild type were transformed with pCM35S-omr1. Ten pots, each with 3-4 plants, were transformed and T1 seeds were harvested from the Tₒ transformed plants of each pot separately. The T1 seeds from each pot were screened for expression of L-O-methylthreonine resistance by germinating in agar medium supplemented with 0.2 mM L-O-methylthreonine, a concentration previously determined and known to completely inhibit the growth of wild type seedlings beyond the cotyledonous stage (Mourad and King, 1995). Half of the T1 seeds from each of the ten pots were screened for L-O-methylthreonine resistance and 5 independent transformants were able to germinate and continue to grow healthy roots and shoots among thousands of seedlings that were completely bleached immediately after the emergence of the cotyledons. In a crowded plate, it is possible to identify the transformants by looking at the bottom of the plate, the transformants show root growth while the nontransformants will have none. After three weeks of growth in the 0.2 mM L-O-methylthreonine agar medium, each of the 5 positive transformants was transferred to soil, kept separately and allowed to self-fertilize to produce the T2 seed.

8. Genetic Characterization of the omr1 Transformants:

The T2 seed was harvested from each of the 5 positive T1 transformants and 50 T2 seeds/transformant were planted in a separate petri plate containing 0.2 mM L-O-methylthreonine agar medium. In each of the 5 petri plates, the majority (75% or more) of the T2 seedlings were resistant to L-O-methylthreonine indicating that a single copy of the transgene omr1 had been inserted in the parent T1 transgenic plant. FIG. 6b shows that 585 amino acid residues of the total 592 residues representing the full length mutant TD were expressed in the transgenic plants. This slightly truncated precursor mutant TD was able to translocate to the chloroplast and confer upon transgenic plants resistance to OMT.

9. Molecular Characterization of the omr1 Transformants:

Two to three leaves of each of the five T1 transformants was excised from the plants at the rosette stage and total DNA was extracted according to a modification of the procedure of Konieczny and Ausubel (1993). A PCR approach was used to confirm the presence of the introduced transgene omr1. For that, a pair of oligonucleotide primers were synthesized such that one primer is complementary to the start of the omr1 and the other primer was complementary to the end of the NOS terminator. The PCR reaction using DNA extracted from each of the five T1 transformants was PCR amplified and each produced a 2.5 kb fragment confirming the presence of the transgene omr1 followed by the NOS terminator in each of the transformants. The native wild type allele OMR1 did not PCR amplify because it is not followed by the NOS terminator and therefore no PCR reaction could take place. DNA extracted from untransformed Arabidopsis plants failed to amplify using such primers.

### EXAMPLE THREE

### The Molecular Basis of L-O-Methylthreonine Resistance Encoded by the omr1 Allele of Line GM11b of Arabidopsis thaliana

1. Isolation of the Wild Type OMR1 Allele:

An Arabidopsis thaliana Columbia wild type cDNA library constructed from 3-day-old seedlings in Stratagene's λ ZAP II vector was screened with a ³²P-labeled 1080 base pair DNA fragment PCR-amplified from the cDNA sequence of omr1 (described above) as a probe. The screening yielded a positive clone TD54 which was purified and was proven to be the wild type allele OMR1 by PCR and Southern analysis.

2. Sequencing of the OMR1 Wild Type Allele:

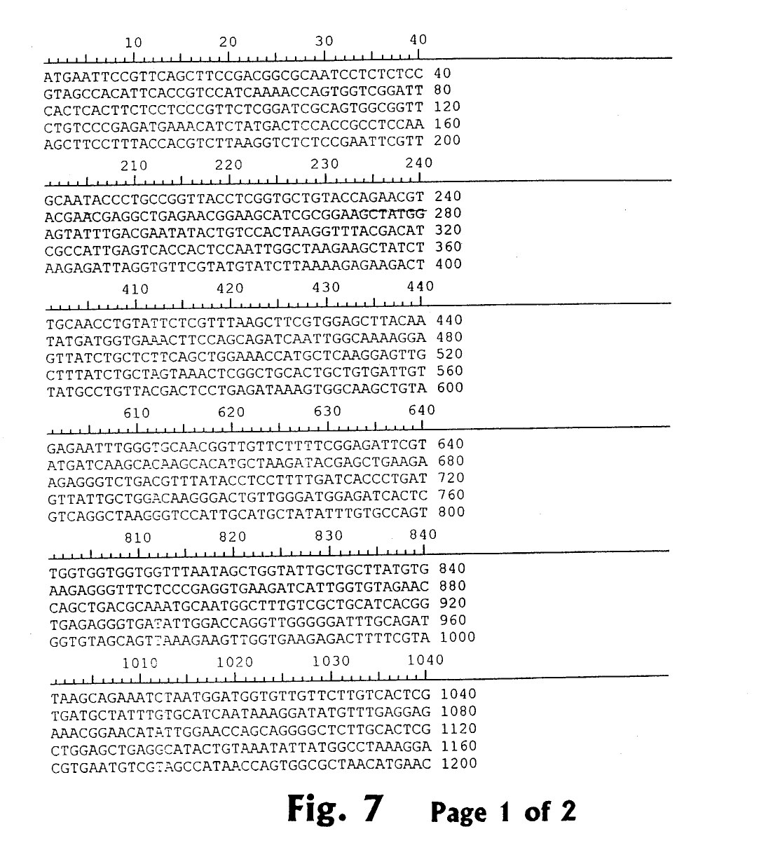

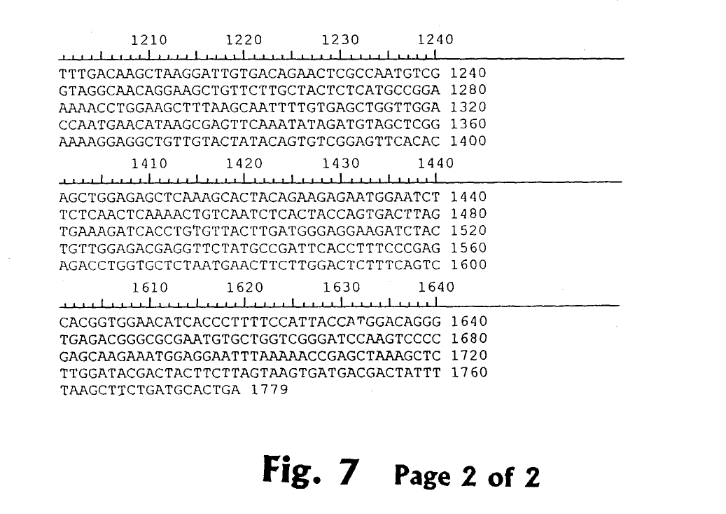

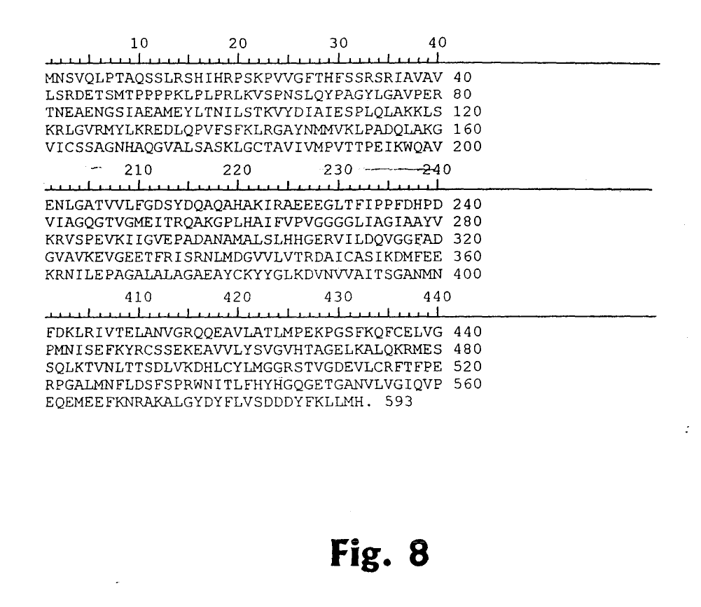

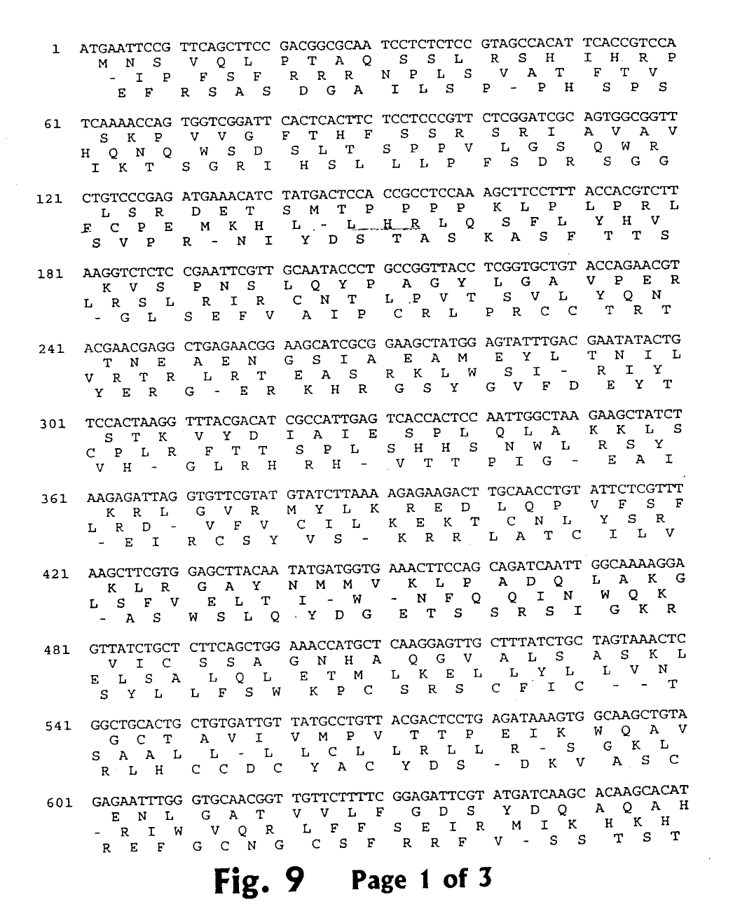

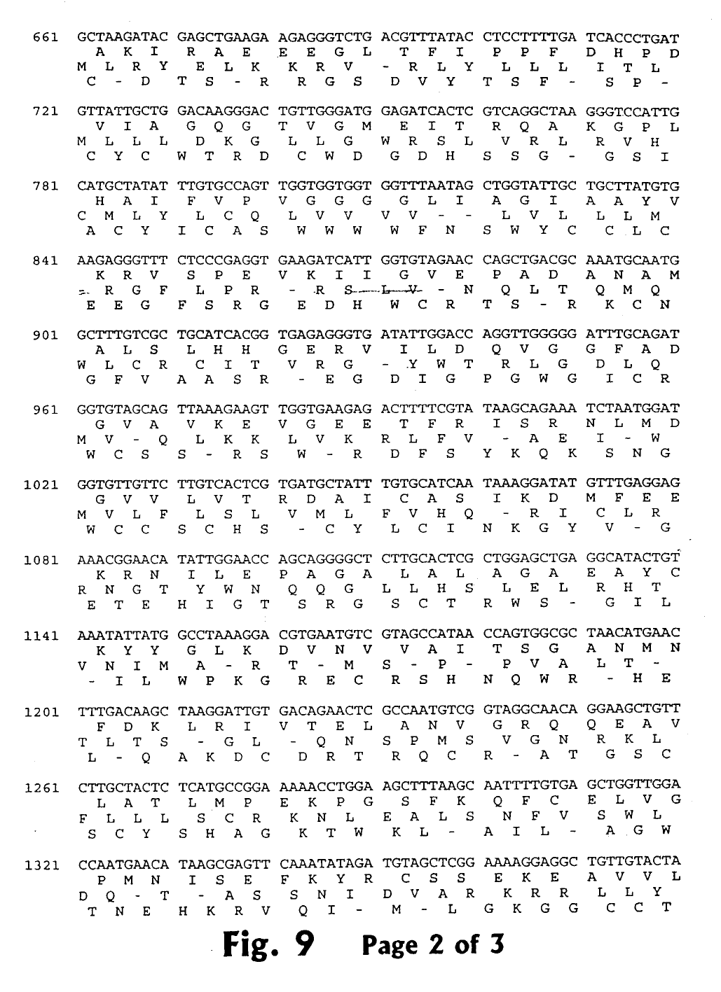

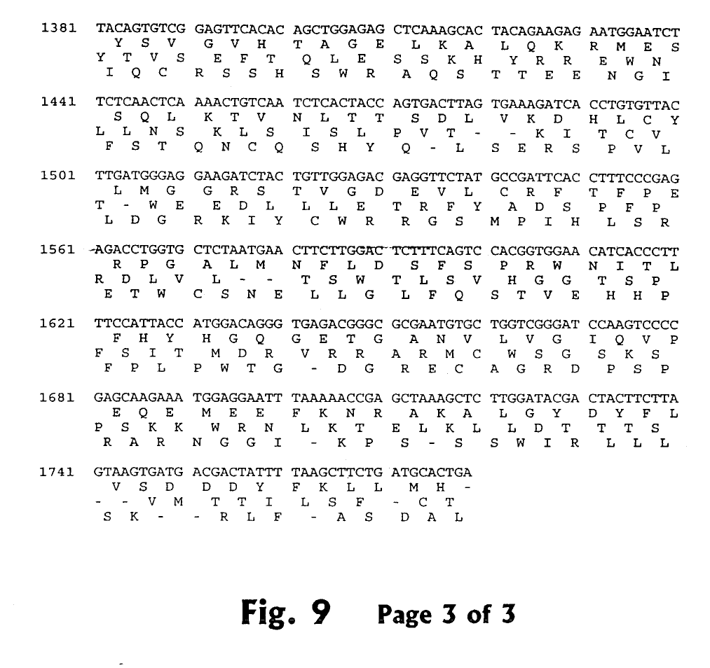

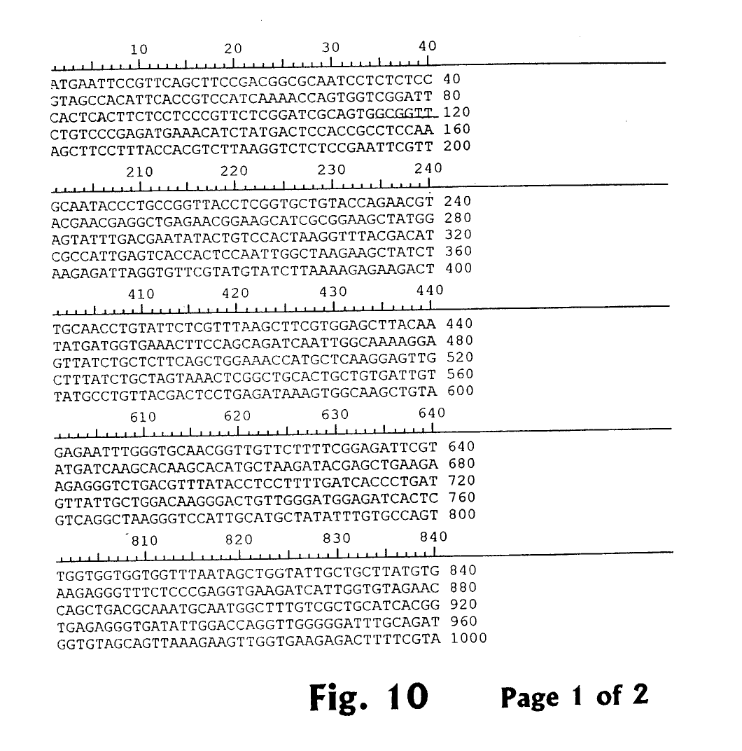

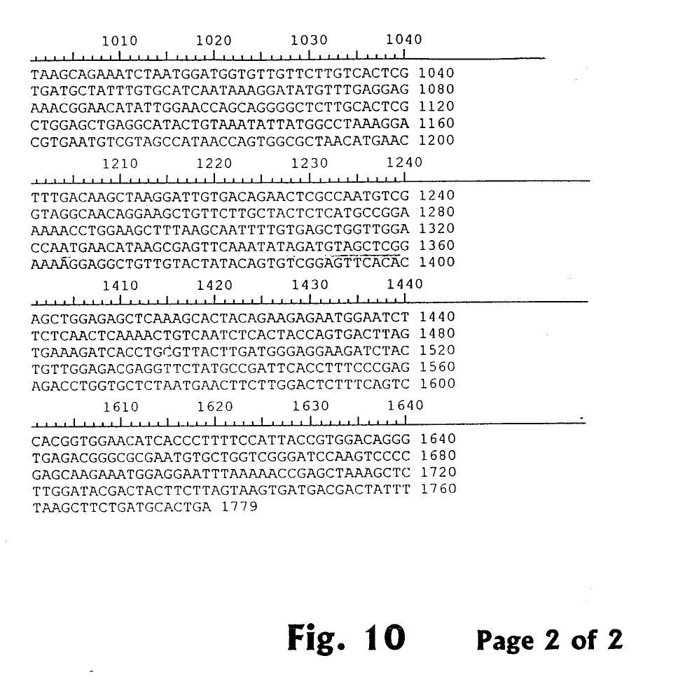

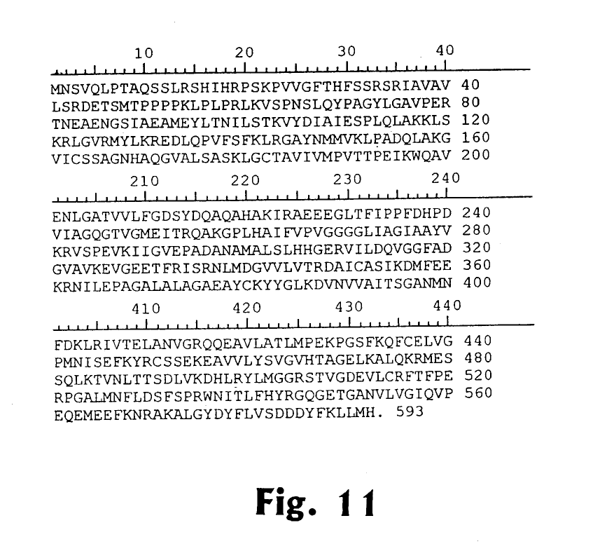

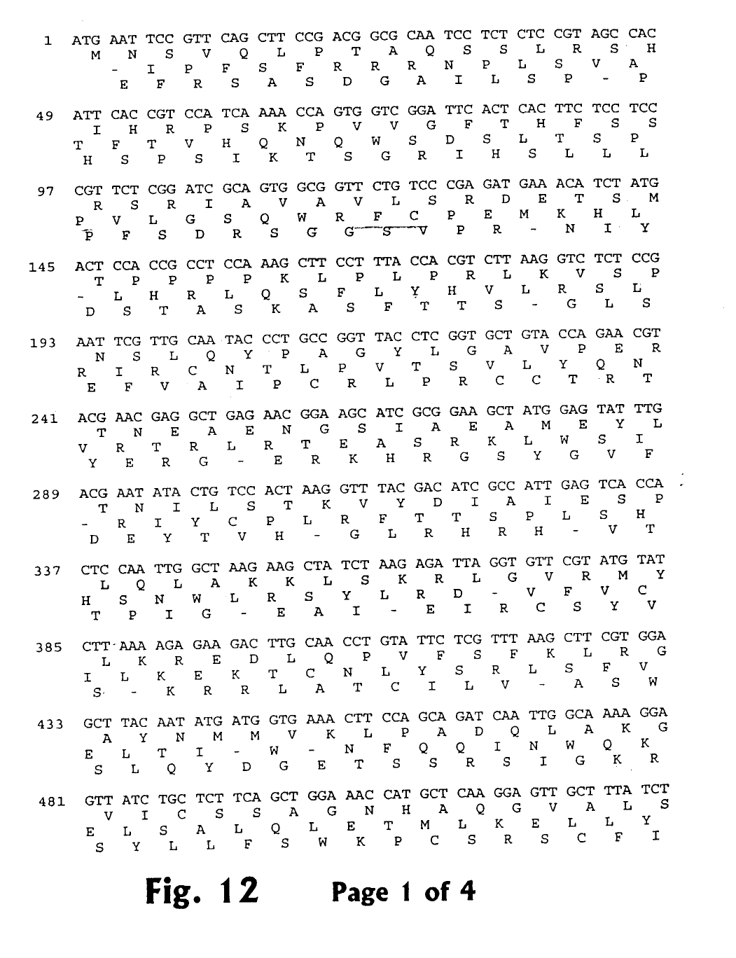

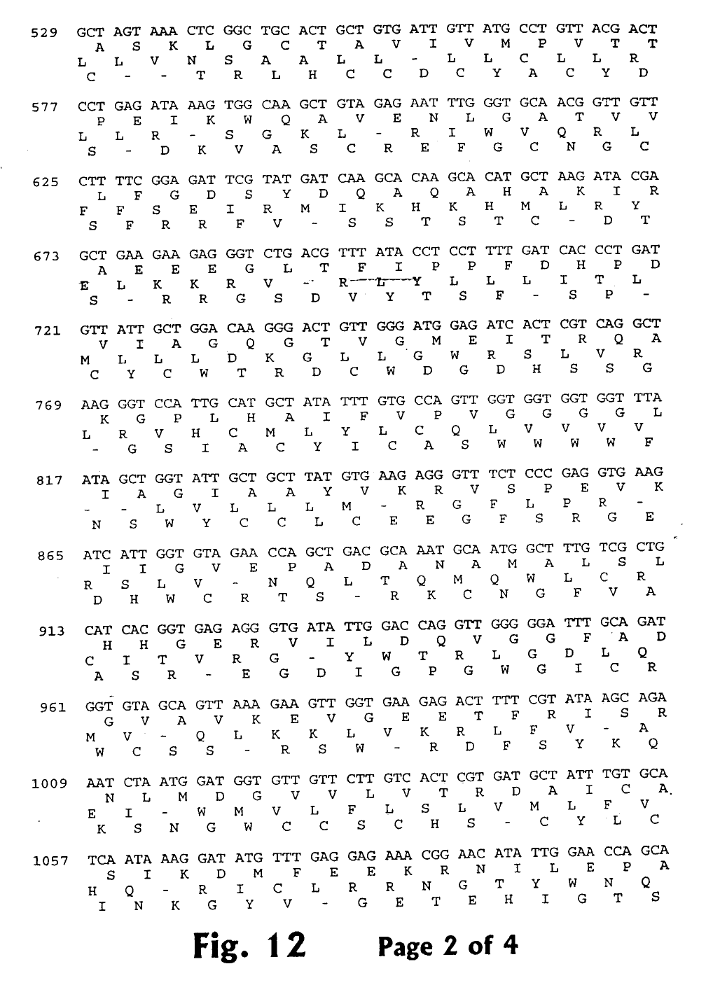

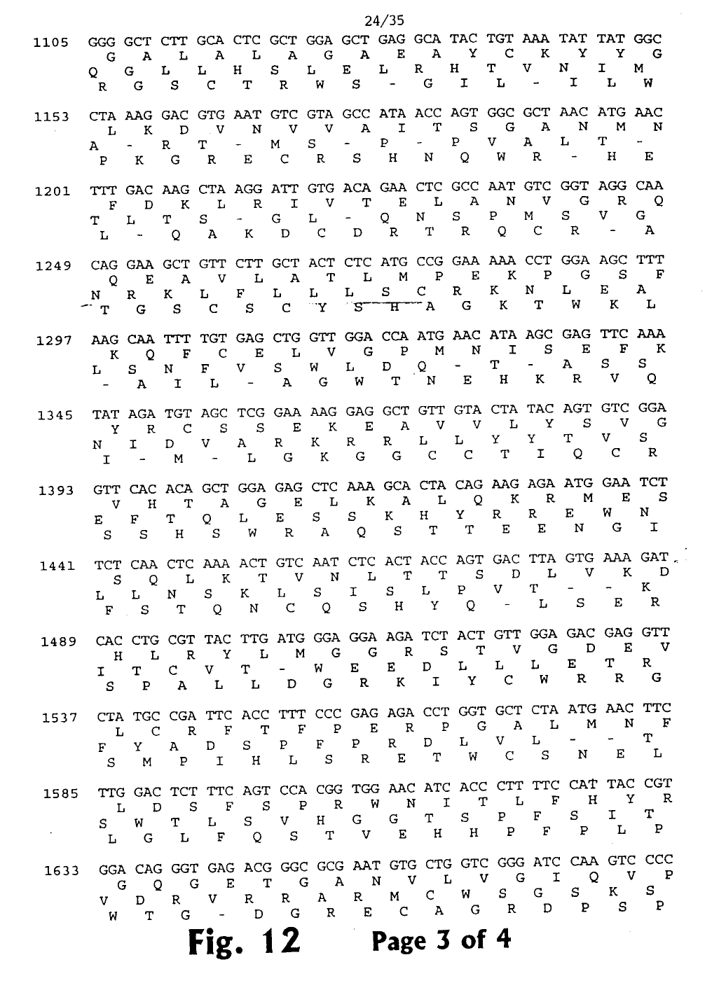

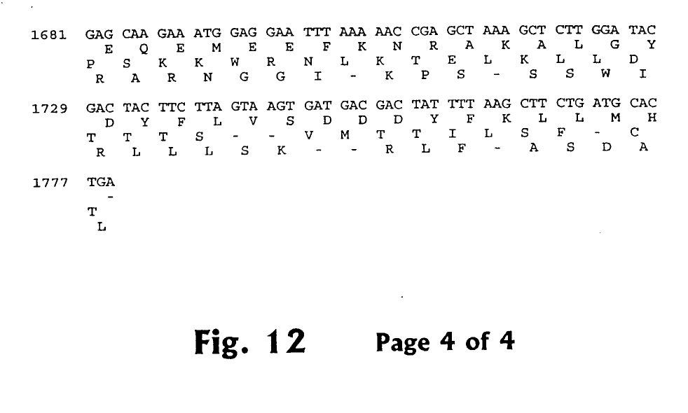

The recombinant plasmid containing the wild type allele OMRI was named pGM-td54 and the OMR1 allele was manually sequenced using the sequenase kit of USB and the same set of oligonucleotide primers that were previously used in sequencing the omr1 allele. The DNA sequence of the wild type OMR1 was similar to that of omr1 except for two different base substitutions predicting two amino acid substitutions in the mutated TD encoded by omr1. In an attempt to clone the 5′ upstream sequences from the ATG start codon of clone 23 (FIG. 5) and using a PCR approach, a new ATG codon was detected at 141 nucleotides upstream from the ATG codon reported in clone 23. This was confirmed in both the wild type allele OMR1 and the mutated allele omr1. Therefore the full length cDNA of the omr1 locus was found to be 1779 nucleotides (FIG. 7) encoding a TD protein of 592 amino acids (FIGS. 8 and 9). The omr1 insert as shown in FIG. 6b (SEQ ID NO:3) was not only strongly expressed in the first transgenic plants (T1) but was also inherited and strongly expressed in their progeny (T2 plants). As expected, the full length cDNA of the OMR1 allele of the omr1 locus was 1779 nucleotides (FIG. 10) encoding a wild type TD of 592 amino acids (FIGS. 11 and 12).

Amino acid alignment of wild type threonine dehydratase/deaminase of Arabidopsis thaliana with that of chickpea (John et al., 1995), tomato (Samach et al., 1991), potato (Hildmann T, Ebneth M, Pena-Cortes H, Sanchez-Serrano J J, Willmitzer L, Prat S (1992) General roles of abscisic and jasmonic acids in gene activation as a result of mechanical wounding. Plant Cell 4:1157-1170.), yeast I (Kielland-Brandt M C, Holmberg S, Petersen J G L, Nilsson-Tillgren T (1984) Nucleotide sequence of the gene for threonine deaminase (ilvl) of Saccharomyces cerevisiae. Carlsberg Res Commun 49:567-575.), yeast 2 (Bornaes C, Petersen J G, Holmberg S (1992) Serine and threonine catabolosm in Saccharomyces cerevisiae: the CHA 1 polypeptide is homologous with other serine and threonine dehydratases. Genetics 131:531-539.), E. coli biosynthetic (Wek R C, Hatfield G C (1986) Nucleotide sequence and in vivo expression of ilvY and ilvC genes in Escherichia coli K12. Transcription from divergent overlapping promoters. J Biol Chem 261:2441-2450.), E. coli catabolic (Datta P, Goss T J, Omnaas J R, Patil R V (1987) Covalent structure of biodegradative threonine dehydratase of Escherichia coli: homology with other dehydratases. Proc Natl Acad Sci USA 84:393-397.), and Salmonella typhimurium (Taillon B E, Little R, Lawther R P (1988) Analysis of the functional domains of biosynthetic threonine deaminase by comparison of the amino acid sequences of three wild type alleles to the amino acid of biodegradative threonine deaminase. Gene 62:245-252.) is set forth in FIG. 13. The Megalign program of the Lasergene software was used, DNASTAR Inc., Madison, Wis. The degree of similarity between amino acid residues of Arabidopsis threonine dehydratase/deaminase and those of other organisms was calculated by the Lipman-Pearson protein alignment method using the Lasergene software and was found to be 46.2% with chickpea, 52.7% with tomato, 55.0% with potato (partial), 45.0% with yeast 1, 24.7% yeast 2, 43.4% with E. coli (biosynthetic), 39.3% with E. coli (catabolic) and 43.3% with Salmonella.

3. Comparing DNA Sequences of omr1 and OMR1 Revealed the Point Mutations Involved:

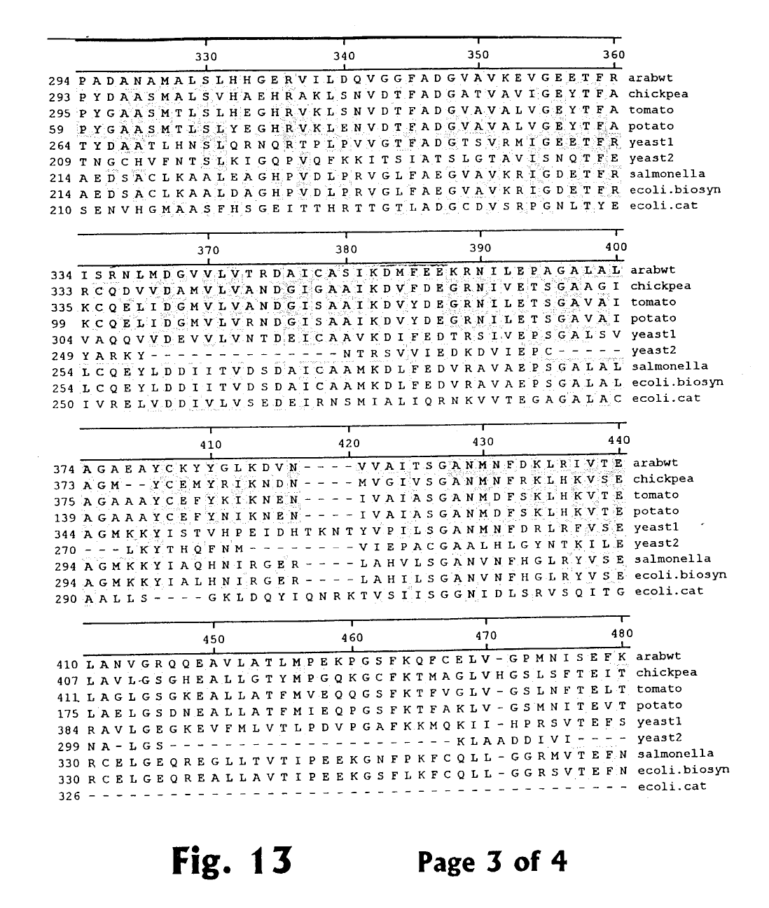

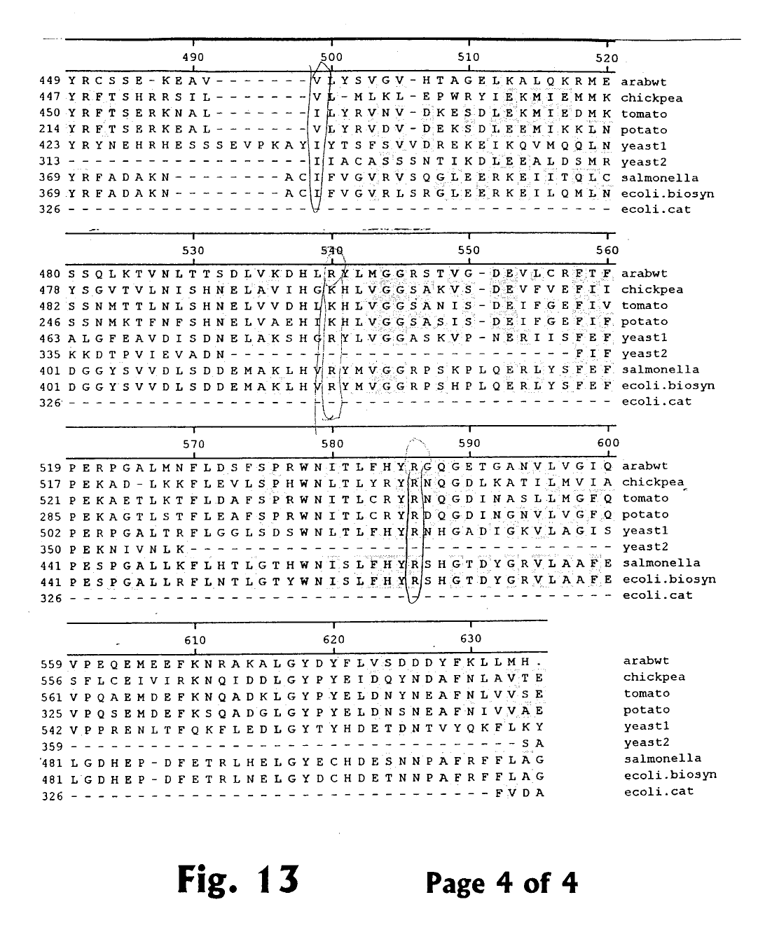

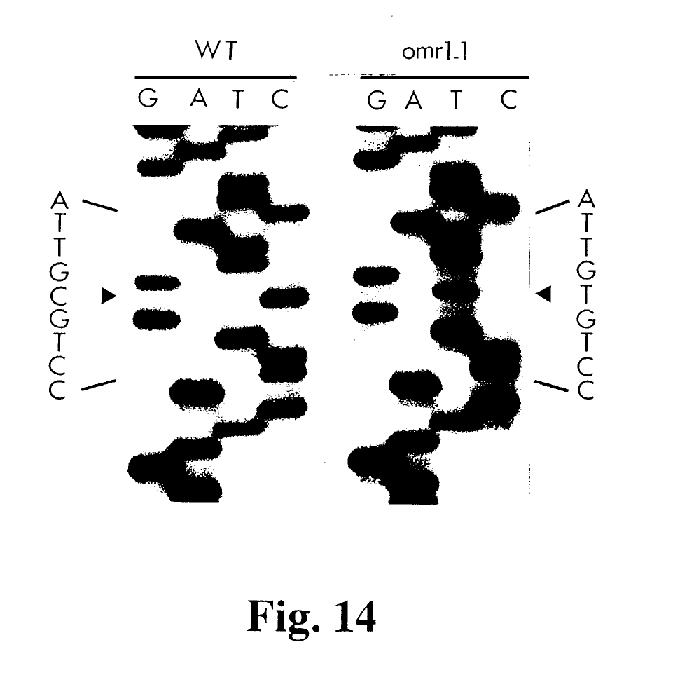

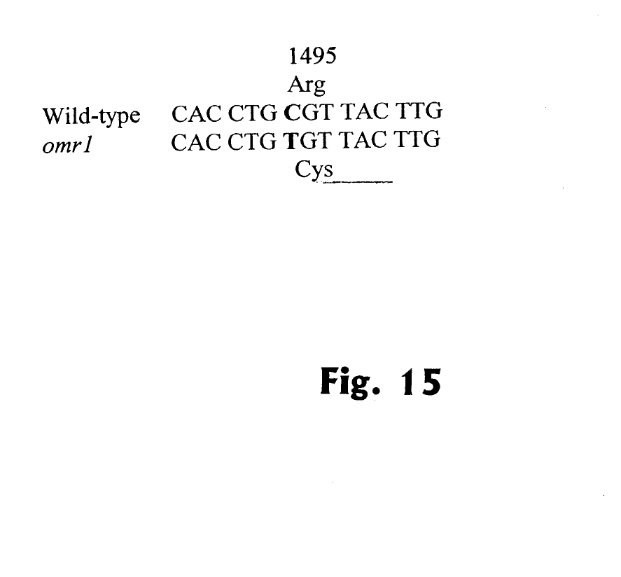

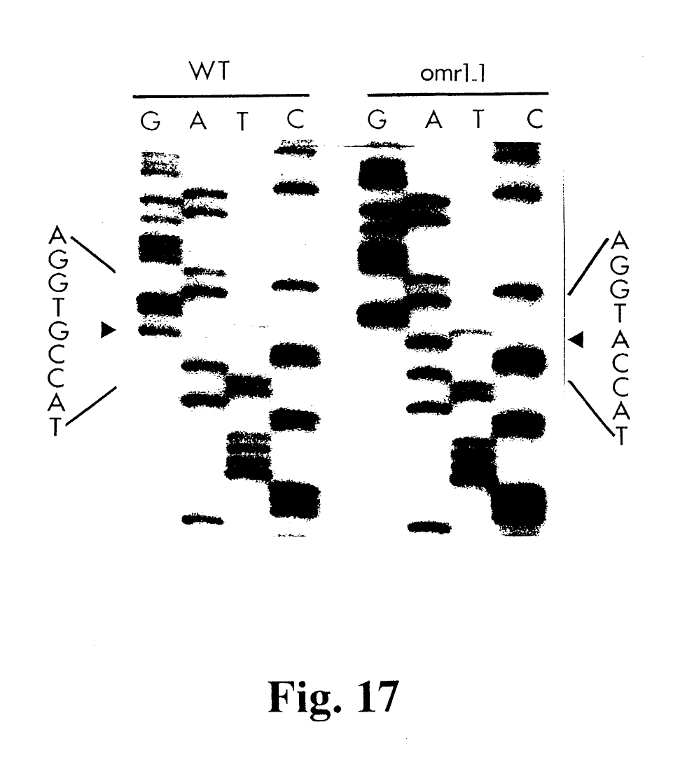

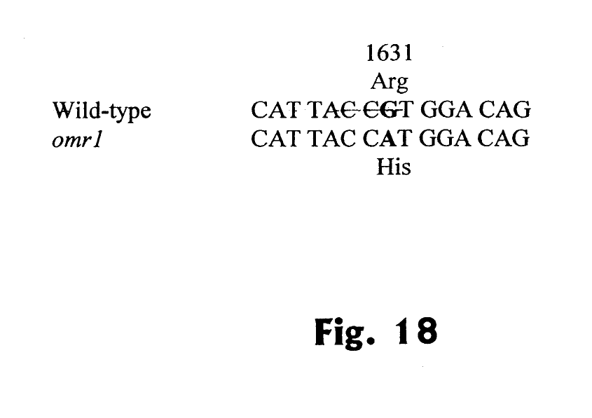

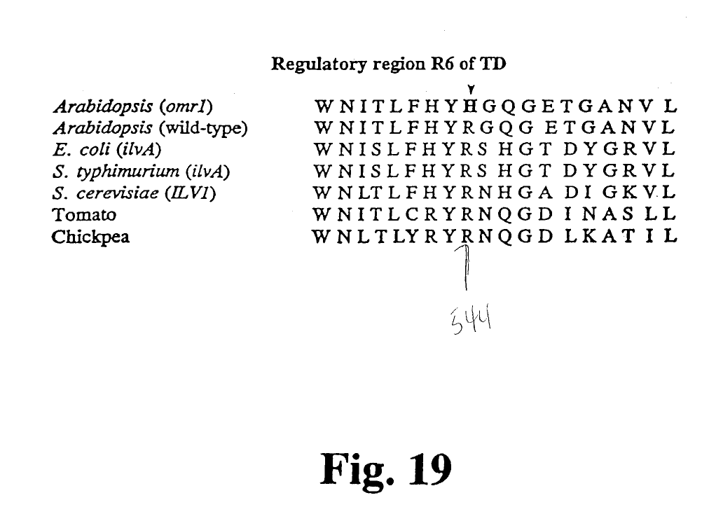

With reference to the nucleotide residue numbering in SEQ ID NO:1 and SEQ ID NO:2, the first base substitution occurred at nucleotide 1519 where C (cytosine) in the wild type allele OMR1 was substituted by T (thymine) in the mutated allele omr1 (FIGS. 14 & 15). This base substitution predicted an amino acid substitution at amino acid residue 452 at the polypeptide level where the arginine residue in the wild type TD encoded by OMR1 was substituted by a cysteine residue in the mutated isoleucine-insensitive TD encoded by omr1 (FIG. 15). This point mutation resides in a conserved regulatory region of amino acids designated R4 (regulatory) by Taillon et al. (1988) where the mutated amino acid is normally an arginine residue in the TD of Arabidopsis, yeast 1, E. coli (biosynthetic) and Salmonella and a lysine residue in the TD of chickpea, tomato, and potato (partial) (FIG. 16). The second base substitution occurred at nucleotide 1655 where G (guanine) in the wild type allele OMR1 was substituted by A (adenine) in the mutated allele omr1 (FIGS. 17 & 18). This base substitution predicted an amino acid substitution at residue 597 at the polypeptide level where the arginine residue in the wild type TD encoded by OMR1 was substituted by a histidine residue in the mutated isoleucine-insensitive TD encoded by omr1 (FIG. 18). This point mutation resides in a conserved regulatory region of amino acids designated R6 (regulatory) by Taillon et al. (1988) where the mutated amino acid is normally an arginine residue in TD of Arabidopsis, chickpea, tomato, potato (partial), yeast 1, E. coli (biosynthetic) and Salmonella (FIG. 19).

What is claimed is:

1. An isolated polynucleotide comprising a nucleotide sequence having substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

2. The polynucleotide in accordance with claim 1, wherein said nucleotide sequence has substantial identity to the sequence set forth in SEQ ID NO:2.

3. The polynucleotide in accordance with claim 1, wherein said nucleotide sequence has substantial identity to the sequence set forth in SEQ ID NO:3.

4. The polynucleotide in accordance with claim 1, wherein said nucleotide sequence has substantial identity to the sequence set forth in SEQ ID NO:4.

5. The polynucleotide in accordance with claim 1, wherein said nucleotide sequence has substantial identity to the sequence set forth in SEQ ID NO:5.

6. The polynucleotide in accordance with claim 1, wherein said nucleotide sequence has substantial identity to the sequence set forth in SEQ ID NO:6.

7. The polynucleotide in accordance with claim 1, wherein said nucleotide sequence has substantial identity to the sequence set forth in SEQ ID NO:7.

8. The polynucleotide in accordance with claim 1, wherein said nucleotide sequence has substantial identity to the sequence set forth in SEQ ID NO:8.

9. The polynucleotide in accordance with claim 1, wherein said nucleotide sequence has substantial identity to the sequence set forth in SEQ ID NO:9.

10. The polynucleotide in accordance with claim 1, wherein said nucleotide sequence has substantial identity to the sequence set forth in SEQ ID NO:10.

11. A polynucleotide comprising a nucleotide sequence selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

12. A polynucleotide having a nucleotide sequence that encodes a functional, feedback-insensitive threonine dehydratase/deaminase enzyme and that hybridizes under moderately stringent conditions with a member selected from the group consisting of the nucleotide sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

13. A nucleotide sequence encoding an amino acid sequence selected from the group consisting of the amino acid sequetce set forth in SEQ ID-NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9, the sequence set forth in SEQ ID NO:10 and amino acid sequences substantially similar thereto.

14. A method for producing cells resistant to structural analogs of isoleucine, comprising:
placing into a cell a construct comprising in the 5′ to 3′ direction of transcription a promoter functional in the cell, a first nucleotide sequence that encodes a transit peptide operably attached to the promoter, a second nucleotide sequence that encodes a mutant, feedback insensitive form of threonine deaminase/dehydratase operably attached to the first sequence, and a termination region functional in the cell operably attached to the second sequence; and
growing the transformed cell whereby the first and second nucleotide sequences are expressed to provide a precursor polypeptide;
wherein expression of the precursor polypeptide allows the cell to be resistant to structural analogs of isoleucine.

15. The method according to claim 14, wherein the precursor polypeptide comprises an amino acid sequence selected from the group consisting of the amino acid sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9, the sequence set forth in SEQ ID NO:10 and amino acid sequences substantially similar thereto.

16. The method according to claim 14, wherein the cell is selected from the group consisting of a plant cell, a bacterial cell, a fungal cell and a yeast cell.

17. A cell produced in accordance with the method of claim 14.

18. A DNA construct comprising a promoter operably linked to a nucleotide sequence encoding a threonine dehydratase/deaminase that is substantially resistant to feedback inhibition.

19. The DNA construct according to claim 18, wherein the nucleotide sequence has substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

20. The DNA construct according to claim 18, wherein the promoter is a plant promoter.

21. The DNA construct according to claim 18, wherein the promoter has substantial identity to a native threonine dehydratase/deaminase promoter.

22. A vector useful for transforming a cell, said vector comprising a nucleotide sequence having substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

23. A plant transformed with the vector of claim 22, or progeny thereof, the plant being capable of expressing the nucleotide sequence.

24. The plant according to claim 23, the plant being selected from the group consisting of gymnosperms, rice, wheat, barley, rye, corn, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

25. A microorganism transformed with the vector of claim 22, or progeny thereof, the microorganism being capable of expressing the nucleotide sequence.

26. The microorganism of claim 25, wherein said microorganism is a yeast cell.

27. The microorganism of claim 25, wherein said microorganism is a bacterial cell.

28. The microorganism of claim 25, wherein said microorganism is a fungal cell.

29. A cell having incorporated therein a foreign nucleotide sequence comprising a promoter operably linked to a nucleotide sequence having substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

30. The cell according to claim 29, wherein the cell is a microorganism.

31. The cell according to claim 29, wherein the cell is a bacterial cell.

32. The cell according to claim 29, wherein the cell is a fungal cell.

33. The cell according to claim 29, wherein the cell is a yeast cell.

34. The cell according to claim 29, wherein the cell is a plant cell.

35. A plant having incorporated into its genome a foreign DNA construct comprising a promoter operably linked to a nucleotide sequence having substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

36. A cell having incorporated into its genome a foreign nucleotide sequence encoding a threonine dehydratase/deaminase that is substantially resistant to feedback inhibition.

37. A method comprising:
incorporating into a plant's genome a DNA construct to provide a transformed plant, the construct comprising a promoter operably linked to a nucleotide sequence having substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10;
wherein the transformed plant is capable of expressing the nucleotide sequence.

38. A method comprising:
providing a vector comprising a promoter operably linked to a nucleotide sequence encoding a threonine dehydratase/deaminase that is resistant to feedback inhibition, wherein the promoter regulates expression of the nucleotide sequence in a host plant cell; and
transforming a target plant with the vector to provide a transformed plant, the transformed plant being capable of expressing the nucleotide sequence.

39. The method according to claim 38, wherein the threonine dehydratase/deaminase comprises an amino acid sequence having substantial similarity to a member selected from the group consisting of the sequence set forth in SEQ ID NO: 2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

40. The method according to claim 38, wherein the nucleotide sequence has substantial identity to the nucleotide sequence of SEQ ID NO:2.

41. A transgenic plant obtained according to the method of claim 38 or progeny thereof.

42. A method for screening potential transformants, comprising:
providing a plurality of cells, wherein at least one of the cells has in its genome an expressible foreign nucleotide sequence having substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10; and
contacting the plurality of cells with a substrate comprising a toxic isoleucine structural analog;
wherein cells comprising the expressible foreign nucleotide sequence are capable of growing in the substrate, and wherein cells not comprising the expressible foreign nucleotide sequence are incapable of growing in the substrate.

43. A method for reliably incorporating a first, expressible, foreign nucleotide sequence into a target cell, comprising:
providing a vector comprising a promoter operably linked to a first primary polynucleotide and a second polynucleotide comprising a nucleotide sequence having substantial identity to a member selected from the group consisting of the sequence set forth in SEQ ID NO: 2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10;
transforming the target cell with the vector to provide a transformed cell; and
contacting the cell with a substrate comprising L-O-methylthreonine;
wherein successfully transformed cells are capable of growing in the substrate, and wherein unsuccessfully transformed cells are incapable of growing in the substrate.

44. A method according to claim 43, wherein the cell is selected from the group comprising a plant cell, a yeast cell, a bacterial cell and a fungal cell.

45. A method for growing a plurality of plants in the absence of undesirable plants, comprising:
providing a plurality of plants, each having in its genome a foreign nucleotide sequence comprising a promoter operably linked to a nucleotide sequence encoding a threonine dehydratase/deaminase that is resistant to feedback inhibition;
growing the plurality of plants in a substrate; and
introducing a preselected amount of an isoleucine structural analog into the substrate.

46. A method according to claim 45, wherein the nucleotide sequence has substantial identity to a member selected from the group consisting of the sequence set forth in SEQ ID NO:2, the sequence set forth in SEQ ID NO:3, the sequence set forth in SEQ ID NO:4, the sequence set forth in SEQ ID NO:5, the sequence set forth in SEQ ID NO:6, the sequence set forth in SEQ ID NO:7, the sequence set forth in SEQ ID NO:8, the sequence set forth in SEQ ID NO:9 and the sequence set forth in SEQ ID NO:10.

47. The method in accordance with claim 45, wherein the analog is L-O-methylthreonine.

48. A method comprising:
providing a nucleotide sequence having substantial identity to the nucleotide sequence set forth in SEQ ID NO:1 or a portion thereof; and
mutating the sequence so that the sequence encodes a feedback insensitive threonine dehydratase/deaminase;
wherein said mutating comprises site-directed mutagenesis.

49. The method according to claim 48, wherein the feedback insensitive threonine dehydratase/deaminase comprises an amino acid other than the wild-type at the amino acid location corresponding to location 452 of SEQ ID NO:2, and at the amino acid location corresponding to location 497 of SEQ ID NO:2.

50. A method comprising:
providing a vector comprising a promoter operably linked to a nucleotide sequence encoding a threonine dehydratase/deaminase that is resistant to feedback inhibition, wherein the promoter regulates expression of the nucleotide sequence in a host cell; and
transforming a target cell with the vector to provide a transformed cell, the transformed cell being capable of expressing the nucleotide sequence.
